(19) United States
(12) Patent Application Publication
(10) Pub. No.: US 2021/0125684 A1
(43) Pub. Date: Apr. 29, 2021

(54) SYSTEMS AND METHODS FOR QUANTITATIVELY PREDICTING RESPONSE TO IMMUNE-BASED THERAPY IN CANCER PATIENTS

(71) Applicant: H. LEE MOFFITT CANCER CENTER AND RESEARCH INSTITUTE, INC., Tampa, FL (US)

(72) Inventor: Joseph MARKOWITZ, Tampa, FL (US)

(21) Appl. No.: 17/047,197

(22) PCT Filed: Apr. 15, 2019
(86) PCT No.: PCT/US2019/027518

§ 371 Date: Oct. 13, 2020

Related U.S. Application Data
(60) Provisional application No. 62/657,273, filed on Apr. 13, 2018.
(60) Provisional application No. 62/686,856, filed on Jun. 19, 2018.
(60) Provisional application No. 62/721,279, filed on Aug. 22, 2018.

(51) Int. Cl. G16B 20/20; G16B 40/00; G16B 45/00; C07K 16/28; G16H 10/40; G16H 10/60; G16H 50/70

(52) U.S. Cl. CPC G16B 20/20; G16B 40/00; G16B 45/00; G16H 50/70; G16H 10/40; G16H 10/60; C07K 16/2818

(57) ABSTRACT

An example method for quantitatively predicting a cancer patient's response to immune-based or targeted therapy is described herein. The method can include receiving patient data for the cancer patient. The patient data is derived from a blood or tissue sample. The method can also include clustering a plurality of immune cell phenotypes present in the patient data, and generating a plurality of violin plots of signal intensity for at least one of the immune cell phenotypes. The clustered patient data can include a plurality of nodes, and each of the violin plots can capture a number of events. The method can further include statistically analyzing the violin plots to predict the cancer patient's response to immune-based or targeted therapy.

### CROSS-REFERENCE TO RELATED APPLICATIONS

This application claims the benefit of U.S. provisional patent application No. 62/657,273, filed on Apr. 13, 2018, and entitled “SYSTEMS AND METHODS FOR QUANTITATIVELY PREDICTING RESPONSE TO IMMUNE-BASED THERAPY IN CANCER PATIENTS,” U.S. provisional patent application No. 62/686,856, filed on Jun. 19, 2018, and entitled “Real-time visual display of multi-dimensional flow cytometry analysis reveals pro- and anti-tumor roles for nitric oxide in melanoma patients receiving adjuvant ipilimumab treatment,” and U.S. provisional patent application No. 62/721,279, filed on Aug. 22, 2018, and entitled “Multi-dimensional flow cytometry analysis reveals pro- and anti-tumor roles for nitric oxide in melanoma patients receiving immunotherapy,” the disclosures of which are expressly incorporated herein by reference in their entireties.

### STATEMENT REGARDING FEDERALLY FUNDED RESEARCH

This invention was made with government support under Grant no. CA168536 awarded by the National Institutes of Health. The government has certain rights in the invention.

### BACKGROUND

One of the most promising areas in cancer research is how the immune system can attack the tumor and hence cure the cancer. Many new “immune therapies” show very promising results when they work but unfortunately ^{˜}60% of melanoma patients do not respond to therapy. Currently there is no test (biomarker) available to find which patients will respond and there is also a lack of understanding why some patients respond while others do not. The immune system consists of many different cell types, such as T cells, macrophages, natural killer cells to name a few. These cells are characterized by expression of certain key proteins (or lack thereof). For example, CD3 expression is specific to T cells. T cells can be further delineated in to CD4 positive cells (T helper cells), and CD8 positive T cells (cytotoxic cells). So to describe a cancer patient's immune system a blood sample with millions of immune cells are used. Each cell is described based on its expression of these immune markers. Standard today is to use more than many types of immune cells markers. It is easy to see that this leads to many thousands of combination of possible cell types and a method to reduce these results down to understandable results is desirable.

### SUMMARY

An example method for quantitatively predicting a cancer patient's response to immune-based or targeted therapy is described herein. The method can include receiving patient data for the cancer patient. The patient data is derived from a blood or tissue sample. The method can also include clustering a plurality of immune cell phenotypes present in the patient data, and generating a plurality of violin plots of signal intensity for at least one of the immune cell phenotypes. The clustered patient data can include a plurality of nodes, and each of the violin plots can capture a number of events. The method can further include statistically analyzing the violin plots to predict the cancer patient's response to immune-based or targeted therapy.

Additionally, the method can optionally include performing immune cell phenotype dimension reduction. In this implementation, the statistically analysis includes analyzing the number of events in each node of the clustered patient data.

Alternatively or additionally, the method can include using the statistical analysis of the violin plots to detect a variation in at least one of the immune cell phenotypes present in the patient data.

Alternatively or additionally, the method can include using the statistical analysis of the violin plots to determine which of the nodes of the clustered patient data are associated with response to immune-based or targeted therapy.

Alternatively or additionally, the method can include using the statistical analysis of the violin plots to determine which of the nodes of the clustered patient data are associated with non-response to immune-based or targeted therapy.

Alternatively or additionally, the statistical analysis can be at least one of a principal component analysis, a cluster analysis technique, a distance matrix analysis, a Cox regression analysis, or a Wilcoxon signed-rank test.

Alternatively or additionally, the violin plots can be generated for each of the nodes of the clustered patient data. In this implementation, each of the violin plots captures the number of events per sample.

Alternatively or additionally, the violin plots can be generated for the blood sample. In this implementation, each of the violin plots captures the number of events per node.

Alternatively or additionally, the method can include generating a graphical display of the clustered patient data. Alternatively or additionally, the method can include generating a graphical display of the violin plots.

Alternatively or additionally, the method can include recommending an immunotherapy or targeted therapy for the cancer patient that is predicted to respond to immune-based or targeted therapy.

Alternatively or additionally, the step of clustering a plurality of immune cell phenotypes present in the patient data can include differentiating between cell populations based on a specific marker. For example, the specific marker can be nitric oxide (NO).

Alternatively or additionally, the immune cell phenotypes present in the patient data can be clustered using at least one of a spanning-tree progression analysis of density-normalized events (SPADE) algorithm, a t-Distributed Stochastic Neighbor Embedding (t-SNE) algorithm, a partitioning algorithm, a hierarchical clustering algorithm, a fuzzy clustering algorithm, a density-based clustering algorithm, or a model-based clustering algorithm.

Alternatively or additionally, the patient data can be at least one of flow cytometry data, immunoassay data (including, but not limited to ELISA, RIA, and ELIspot), microscopy image data (including, but not limited to IHC or immunofluorescence staining of tissue slides (FFPE and fresh-frozen specimens)), mass spectrometry data, mass cytometry data, or genomic data.

Alternatively or additionally, the immune cell phenotypes can include myeloid markers, which can include, but are not limited to, HLA-DR, CD33, CD16, CD44, CD66, Cd1c, CD83, CD141, CD209, MHC II, CD123, CD303, CD304, CD34, CD90, CD68, CD163, CD64, CD49d, 2D7 antigen, CD123, CD203c, FcεRIg, CD193, EMR1, Siglec-8, PD-1, PD-L1, Tim3, CD138, CD45, CD117, CD11b, CD34, CD36, CD64, CD61, CD117, CD62L, CD14, CD15, CD11c, CD103, DAF-FM, CTLA-4, FOXP3, Arginase I, or IFN-γ.

Alternatively or additionally, the immune cell phenotypes can include lymphoid markers, which can include, but are not limited to, CD3, CD3z, CD4, CD8, CD56, CD25, CD69, CD138, CD27, CD44, NKG2D, NKp30, NKp46, NKp46, CTLA-4, LaG-3, PD-1, TIM-3, PD-L1, CD45RA, CD45RO, CD62L, CD69, CD127, CD19, CD11c, CCR7, CTLA-4, or DAF-FM as well as intracellular markers such as CTLA-4, FOXP3, Arginase I, and IFN-γ.

Alternatively or additionally, the cancer patient can have lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. In one aspect, the patient can have melanoma.

An example method for treating a cancer patient is also described herein. The method can include predicting the cancer patient's response to immune-based or targeted therapy as described herein, and administering an immunotherapy or targeted therapy to the cancer patient that is predicted to respond to immune-based or targeted therapy.

An example system for quantitatively predicting a cancer patient's response to immune-based or targeted therapy is also described herein. The system can include a processor, and a memory operably coupled to the processor. The memory can have computer-executable instructions stored thereon that, when executed by the processor, cause the processor to receive patient data for the cancer patient, cluster a plurality of immune cell phenotypes present in the patient data, generate a plurality of violin plots of signal intensity for at least one of the immune cell phenotypes, and statistically analyze the violin plots to predict the cancer patient's response to immune-based or targeted therapy.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating example operations for predicting a cancer patient's response to immune-based therapy.

FIGS. 2A-2E illustrate the multidimensional phenotyping analysis tool in R (MPAT-R) algorithm used to phenotype patient samples according to an implementation described herein. FIG. 2A illustrates clustering of immune cell phenotypes. FIGS. 2B and 2C illustrate violin plots. FIG. 2D illustrates phenotype dimension reduction, i.e., the schema for the number of events per node versus patient sample. FIG. 2E is a flow chart illustrating example operations for the MPAT-R algorithm.

FIG. 3 is an example computing device.

FIG. 4 is a table illustrating example myeloid markers.

FIG. 5 is a table illustrating example lymphoid markers.

FIG. 6 is a table illustrating example myeloid FSC and SSC nodes.

FIG. 7 is a table illustrating example lymphoid FSC and SSC nodes.

FIGS. 8A-8C illustrate the multidimensional phenotyping analysis tool in R (MPAT-R) algorithm used to phenotype patient samples according to another implementation described herein. FIG. 8A illustrates clustering of immune cell phenotypes. FIGS. 8B and 8C illustrate violin plots.

FIGS. 9A-9H illustrate the integration of Multi-Dimensional Phenotype Analysis Tool in R (MPAT-R) output for lymphoid markers and delineation of cellular subsets associated with relapse-free survival (RFS) or treatment effects according to an implementation described herein.

FIGS. 10A-10F illustrate the integration of the Multi-Dimensional Phenotype Analysis Tool in R (MPAT-R) output for the myeloid markers and delineation of cellular subsets associated with relapse-free survival (RFS) or treatment effects according to an implementation described herein.

FIGS. 11A-11H illustrate Kaplan Meier survival curves illustrating types of relationships between immune cell subsets and relapse free survival (strata=≤median number of events/node, >median number of events/node) according to an implementation described herein.

FIGS. 12A-12B illustrate the dichotomous role for nitric oxide (NO) in pro- and anti-tumor effects.

FIG. 13 illustrates phospho-STAT1 expression in patients with an RFS less than one year and patients with an RFS greater than one year. pSTAT1 increases in peripheral blood immune cells in patients with long RFS. RFS greater than 1 year (Mean=1022.45; standard deviation (SD)=277.05); RFS less than or equal to 1 year (Mean=773.12; SD=306.18); p=0.001.

FIG. 14A illustrates nitric oxide levels in immune cell subsets associated with adjuvant ipilimumab and peptide vaccine treatment, identified by the MPATR algorithm. FIG. 14B illustrates nitric oxide levels in immune cell subsets that changed with adjuvant ipilimumab and peptide vaccine treatment but were not associated with RFS, identified by the MPATR algorithm.

FIG. 15 is Table S1, which illustrates clinical characteristics of metastatic melanoma patients who received adjuvant ipilimumab and peptide vaccine treatment.

FIG. 16 is Table S2, which illustrates antibodies utilized for the flow cytometry analysis of patient samples.

FIG. 17 is Table S3, which illustrates lymphoid phenotypes for statistically significant nodes generated from the MPATR analysis.

FIG. 18 is Table S4, which illustrates lymphoid phenotypes for statistically significant nodes that were generated from the MPATR analysis using the addition of the scatter properties forward scatter area and side scatter area (FSC and SSC).

FIG. 19 is Table S5, which illustrates myeloid phenotypes for statistically significant nodes generated from the MPATR analysis.

FIG. 20 is Table S6, which illustrates myeloid phenotypes for statistically significant nodes that were generated from the MPATR analysis using the addition of the scatter properties forward scatter area and side scatter area (FSC and SSC).

FIGS. 21A-21C illustrate that patients responding to anti-PD-1 have increased expression of PD-L1 on melanoma tumor cells (SOX10+) and infiltrating T cells.

FIGS. 22A-22C illustrate the multidimensional phenotyping analysis tool in R (MPAT-R) algorithm used to phenotype patient samples according to another implementation described herein. FIG. 22A illustrates clustering of immune cell phenotypes. FIGS. 22B and 22C illustrate violin plots.

FIGS. 23A-23F illustrate a principal component (PCA) analysis with SPADE trees for example lymphoid markers. FIGS. 23A-23B are a pre-treatment analysis, and FIGS. 23C-23D are a post-treatment analysis, and FIGS. 23E-23F are a combined pre- and post-treatment analysis.

FIGS. 24A-24F illustrate PCA analysis with SPADE trees for example myeloid markers. FIGS. 24A-24B are a pre-treatment analysis, and FIGS. 24C-24D are a post-treatment analysis, and FIGS. 24E-24F are a combined pre- and post-treatment analysis.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for quantitatively predicting response to immune-based therapy for a patient with melanoma, it will become evident to those skilled in the art that the implementations are not limited thereto, but are applicable for quantitatively predicting response to targeted therapy for melanoma.

### EXAMPLE EMBODIMENTS

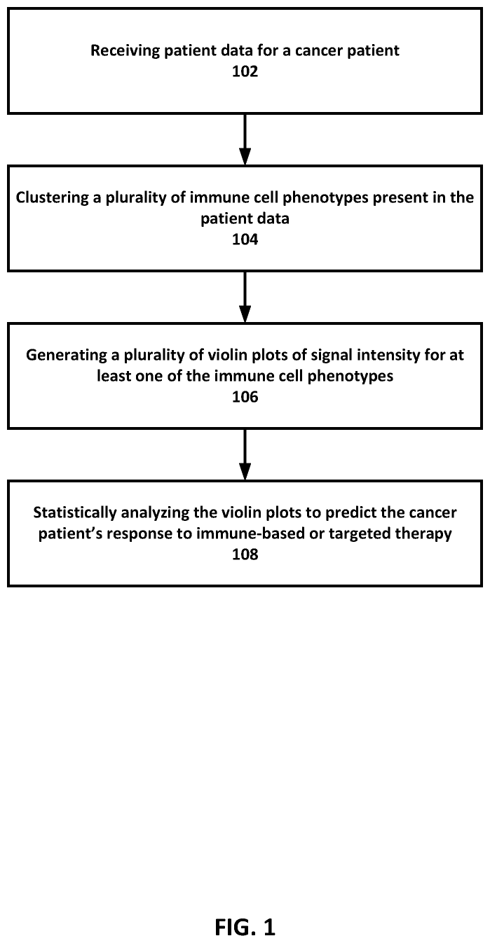

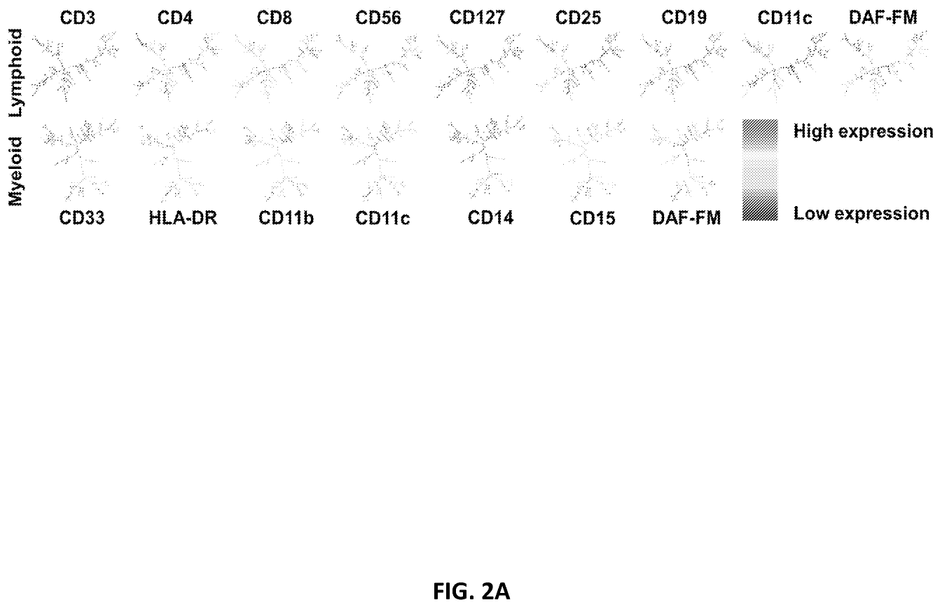

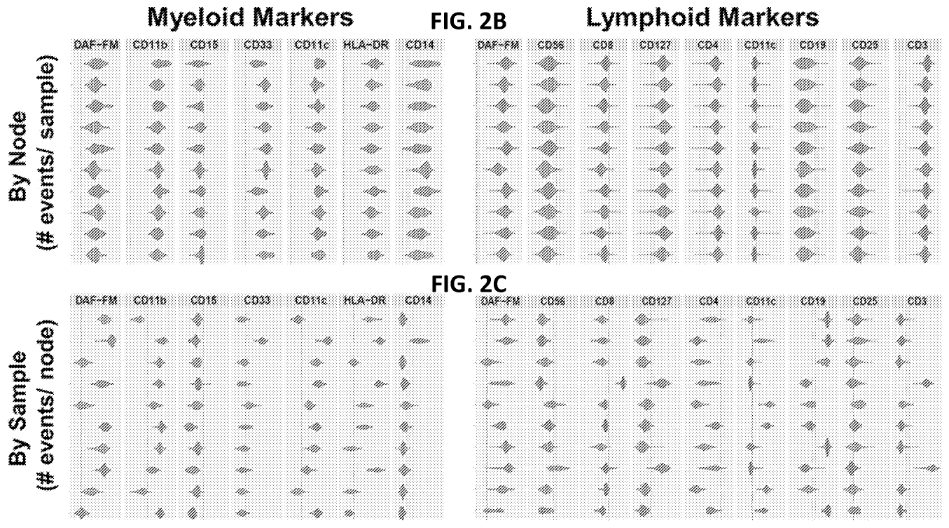

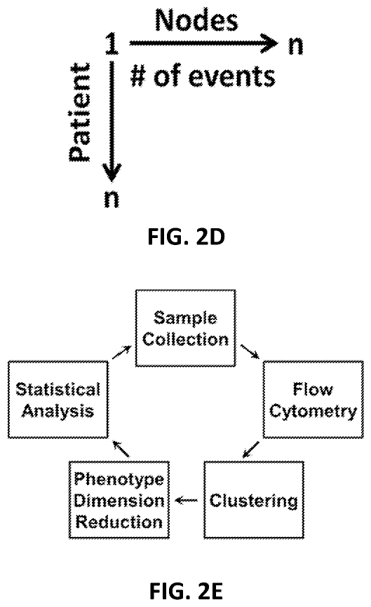

Referring now to FIGS. 1-2E, an example method for quantitatively predicting a cancer patient's response to immune-based or targeted therapy is described. As described herein, peripheral blood contains many types of immune cells that can be monitored and demonstrate change with therapy. Conventionally, high-dimensional flow cytometry phenotyping can be performed and analyzed via clustering algorithms including SPADE (Spanning-tree Progression Analysis of Density-normalized Events), t-SNE (t-Distributed Stochastic Neighbor Embedding), and viSNE (visualization tool for t-SNE). However, the outputs of these clustering algorithms require manual curation based on marker expression for individual cells. This process is not user friendly and is also time consuming. The Multi-Dimensional Phenotyping Analysis Tool described herein overcomes the limitations of these clustering algorithms. As described below, the Multi-Dimensional Phenotyping Analysis Tool facilitates high-dimensional phenotyping of cell types (e.g., immune cell phenotypes), the results of which (e.g., generated violin plots and/or statistical analysis of the same) can be easily interpreted by clinicians. For example, as described below, violin plots of signal intensity for the immune cell phenotypes can be generated following application of a clustering algorithm to the immune cell phenotypes. The violin plots can then be statistically analyzed to predict a patient's response to immunotherapy. Optionally, a clinician can use this information to recommend and/or administer immunotherapy to the patient. In other words, the Multi-Dimensional Phenotyping Analysis Tool described herein has advantages as compared to conventional high-dimensional flow cytometry phenotyping techniques.

In the examples described below, the cancer patient has melanoma. It should be understood that melanoma is provided only as an example and that the patient can have other cancers. For example, the patient may have another cancer that resists immune-based therapy. For example, the cancer patient can have lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. The example method can be used to predict which patients are likely to respond (or fail to respond) to immune-based therapy such as anti-PD-1 therapy. The ability to make such a prediction can allow a patient predicted to fail anti-PD-1 therapy to be treated with other potentially more effective therapies such as combining anti-PD-1 with nitro-aspirin. Additionally, the ability to make such a prediction can allow spare a patient predicted to fail anti-PD-1 therapy exposure to unnecessary toxicity.

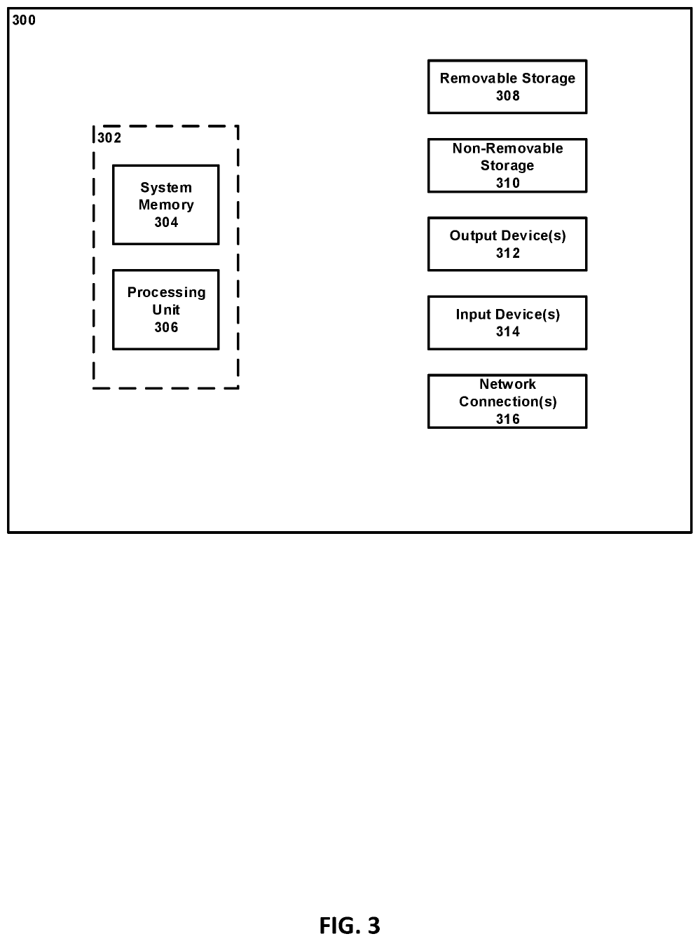

The method can include receiving patient data for the cancer patient (e.g., FIG. 1, Step 102). Optionally, the cancer patient has not yet received immunotherapy treatment. The patient data can be derived from a blood or tissue sample or specimen taken from the cancer patient (e.g., a peripheral blood sample from a metastic melanoma patient). In some implementations, the patient data can be flow cytometry data as described in the examples below. For example, the flow cytometry data can be used to measure nitric oxide (NO) levels within myeloid and lymphocyte subsets (see FIGS. 2A-2C). It should be understood that flow cytometry data is provided only as an example and that the patient data can be other types of data including, but not limited to, immunoassay data (including, but not limited to ELISA, RIA, and ELIspot), microscopy image data (including, but not limited to immunofluorescence data), mass spectrometry data, mass cytometry data, or genomic data. This disclosure contemplates that the patient data can be received at a computing device such as computing device 300 shown in FIG. 3.

Optionally, the patient data can transmitted over a network by a remote computing device (e.g., a flow cytometer and/or remote server) and then received by the computing device. The computing devices discussed above can be connected by one or more networks. This disclosure contemplates that the networks are any suitable communication network. The networks can be similar to each other in one or more respects. Alternatively or additionally, the networks can be different from each other in one or more respects. The networks can include a local area network (LAN), a wireless local area network (WLAN), a wide area network (WAN), a metropolitan area network (MAN), a virtual private network (VPN), etc., including portions or combinations of any of the above networks. The computing devices discussed above can be coupled to the networks through one or more communication links. This disclosure contemplates the communication links are any suitable communication link. For example, a communication link may be implemented by any medium that facilitates data exchange between the computing devices including, but not limited to, wired, wireless and optical links. Example communication links include, but are not limited to, a LAN, a WAN, a MAN, Ethernet, the Internet, or any other wired or wireless link such as WiFi, WiMax, 3G or 4G.

The method can also include clustering a plurality of immune cell phenotypes present in the patient data (e.g., FIG. 1, Step 104). This disclosure contemplates that clustering can be performed at a computing device such as computing device 300 shown in FIG. 3. As described herein, the step of clustering a plurality of immune cell phenotypes present in the patient data can include differentiating between cell populations based on a specific marker such as nitric oxide (NO). As described herein, the ability of melanoma to resist immune-based therapy is related to the level of NO, e.g., immune suppressor cells such as myeloid-derived suppressor cells (MDSCs) release large amounts of NO, which can inactivate proteins such as STAT1 that normally help immune cells sense and respond to cancer cells. NO can therefore serve as the marker for differentiating cell populations. It should be understood that NO is provided only as an example and that other markers relevant to immune-based therapy resistance may be used. Optionally, the immune cell phenotypes present in the patient data can be clustered using a spanning-tree progression analysis of density-normalized events (SPADE) algorithm. SPADE is an algorithm that can be used to organize cells into hierarchies of related phenotypes. The SPADE algorithm is known in the art and is therefore not described in further detail herein. For example, the SPADE algorithm is described in P. Qiu, E. F. Simonds, S. C. Bendall, K. D. Gibbs, Jr., R. V. Bruggner, M. D. Linderman, K. Sachs, G. P. Nolan, S. K. Plevritis, Extracting a cellular hierarchy from high-dimensional cytometry data with SPADE, Nat Biotechnol, 29 (2011) 886-891. It should be understood that the SPADE algorithm is provided as an example clustering algorithm and that other clustering algorithms such as t-SNE (t-Distributed Stochastic Neighbor Embedding), viSNE (visualization tool for t-SNE), partitioning methods, hierarchical clustering, fuzzy clustering, density-based clustering, or model-based clustering, can be used.

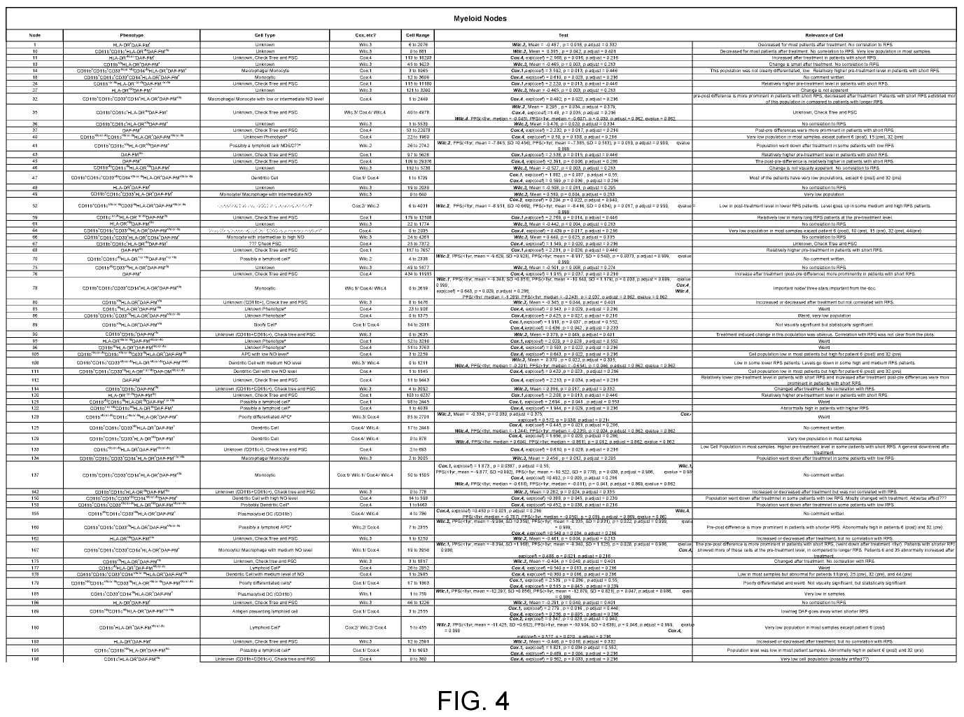

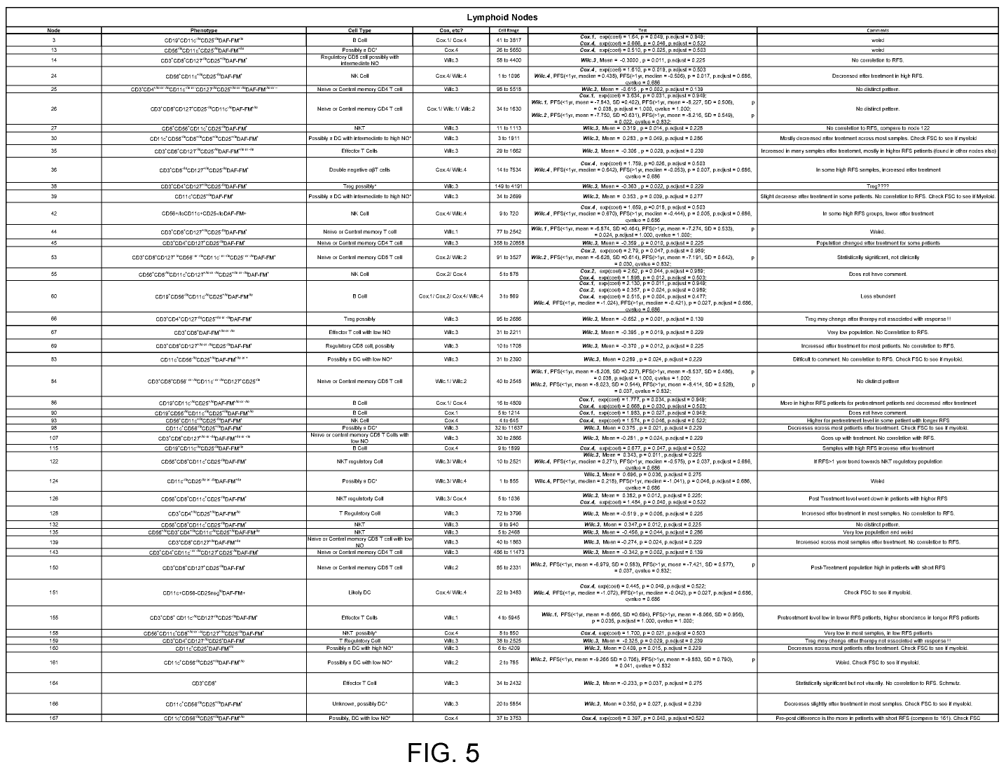

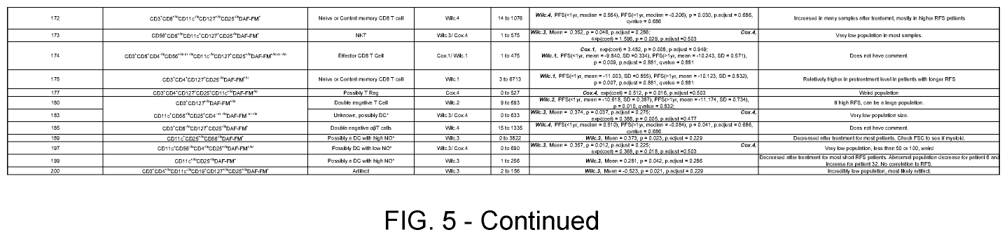

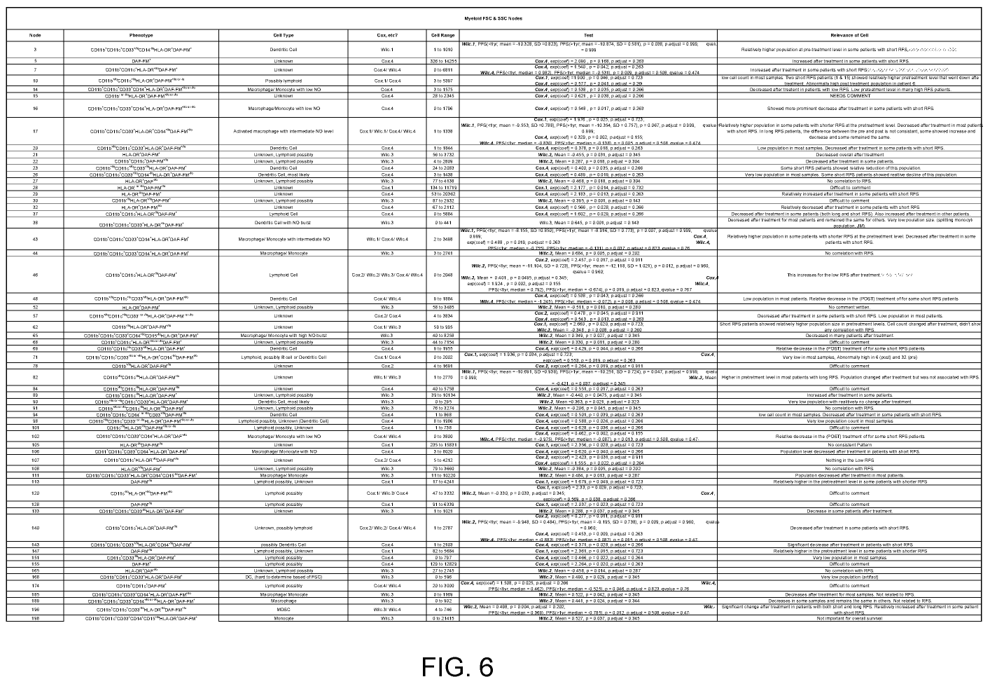

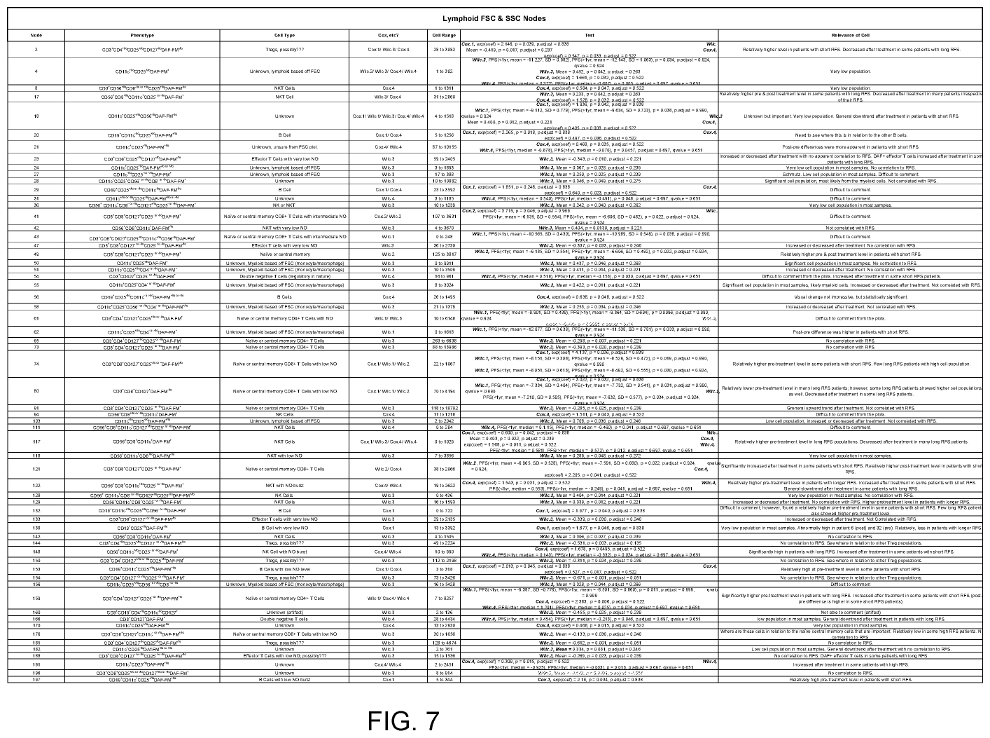

Example results of clustering a plurality of immune cell phenotypes present in the patient data are shown in FIGS. 2A, 8A and 22A. As shown in FIGS. 2A, 8A, and 22A, a spanning tree is shown for each of seven different myeloid markers and nine different lymphoid markers. Each spanning tree includes a plurality of nodes, which are color coded according to the NO expression level. The immune cell phenotypes can include myeloid markers, which can include, but are not limited to, HLA-DR, CD33, CD16, CD44, CD66, Cd1c, CD83, CD141, CD209, MHC II, CD123, CD303, CD304, CD34, CD90, CD68, CD163, CD64, CD49d, 2D7 antigen, CD123, CD203c, FcεRIg, CD193, EMR1, Siglec-8, PD-1, PD-L1, Tim3, CD138, CD45, CD117, CD11b, CD34, CD36, CD64, CD61, CD117, CD62L, CD14, CD15, CD11c, CD103, DAF-FM, CTLA-4, FoxP3, Arginase I, or IFN-γ. For example in one implementation, the myeloid markers can include CD11c, CD11b, CD33, HLA-DR, CD14, CD15, PD-L1, CTLA-4, FoxP3, Arginase I, and IFN-γ. Alternatively or additionally, this disclosure contemplates that tumor specific markers and/or targeted antibodies can also be used. In the case of melanoma, tumor specific markers include SOX-10, S100, MART-1/Melan-A, tyrosinase, MITF. Targeted antibodies can also be used for BRAF V600E, NRAS, STAT proteins, other HLA molecules, STING, IRF3, pIRF3. Additionally, the immune cell phenotypes can include lymphoid markers, which can include, but are not limited to, CD3, CD3z, CD4, CD8, CD56, CD25, CD69, CD138, CD27, CD44, NKG2D, NKp30, NKp46, NKp46, CTLA-4, LaG-3, PD-1, TIM-3, PD-L1, CD45RA, CD45RO, CD62L, CD69, CD127, CD19, CD11c, CCR7, CTLA-4, or DAF-FM as well as intracellular markers such as CTLA-4, FOXP3, Arginase I, and IFN-γ. For example, in one implementation, the lymphoid markers can include CD11c, CD3, CD4, CD8, CD25, CD127, CD19, CD56, CD69, PD-L1, CTLA-4, CD3z, FoxP3, Arginase I, or IFN-γ. Alternatively or additionally, platelets and RBC can also be determined via CD42b, CD62P, CD235a. This disclosure contemplates that the immune cell phenotypes can include markers other than myeloid and/or lymphoid cells. Additionally, it should be understood that the specific myeloid and/or lymphoid markers shown in FIGS. 2A, 8A, and 22A are provided as examples only and that other phenotypes can be used. For example, the other phenotypes can include, but are not limited, to those shown in FIG. 4 (myeloid phenotype table) and FIG. 5 (lymphoid phenotype table).

The method can also include generating a plurality of violin plots of signal intensity for at least one of the immune cell phenotypes (e.g., FIG. 1, Step 106). This disclosure contemplates that the violin plots can be generated using a computing device such as computing device 300 shown in FIG. 3. Optionally, the method can include generating a graphical display of the clustered patient data (see FIGS. 2A, 8A, and 22A). Alternatively or additionally, the method can optionally include generating a graphical display of the violin plots (see FIGS. 2B, 2C, 8B, 8C, 22B, and 22C). The graphical display of the violin plots allows a user (e.g., a clinician) to view all of the phenotypes across all of the markers of interest (e.g., nodes and/or samples) and all of the patients simultaneously. This disclosure contemplates displaying the graphical display on a display device (e.g., output device 312 of FIG. 3). Example violin plots are shown in FIGS. 2B, 2C, 8B, 8C, 22B, and 22C. A violin plot illustrates the signal intensity (e.g., signal intensity of a fluorophore) and captures the number of events. As used herein, each event is a cell that is measured with a particular phenotype. A violin plot is constructed by plotting the signal intensity of each antibody on each cell (event). The cut-offs are constructed by using the Fluorescence minus one and/or Isotype antibody controls. In FIGS. 2B and 2C, the 99% and 95% cut-off of the Isotype antibody controls were utilized. It should be understood that the cut-off values provided above are only examples. This disclosure contemplates that user-defined (e.g., experimentally obtained) cut-offs can be used for displaying data. In experiments, the Fluorescence minus one (FMO) and Isotype antibody controls were shown to be roughly equivalent for this experiment. In one implementation, the violin plots can be generated for each of the nodes of the clustered patient data. This is shown in FIGS. 2B, 8B, and 22B. In this implementation, each of the violin plots captures the number of events per blood sample. In FIGS. 2B, 8B, and 22B, each of the columns represents one of the myeloid and lymphoid markers, and each of the rows represents a node. In another implementation, the violin plots can be generated for a sample (e.g., blood sample). This is shown in FIGS. 2C, 8C, and 22C. In this implementation, each of the violin plots captures the number of events per node. In FIGS. 2C, 8C, and 22C, each of the columns represents one of the myeloid and lymphoid markers, and each of the rows represents a sample.

The method can further include statistically analyzing the violin plots to predict the cancer patient's response to immune-based therapy (e.g., FIG. 1, Step 108). As described herein, a statistical analysis can be used to evaluate which populations (e.g., nodes/samples) are associated with an outcome (e.g., RFS). This disclosure contemplates that the statistical analysis can be performed using a computing device such as computing device 300 shown in FIG. 3. For example, a data set with patient data for a plurality of patients can be compiled. This patient data can optionally be retrieved over a network from one or more databases and/or electronic medical records. This disclosure contemplates that the data set can be stored in memory of a computing device such as computing device 300 shown in FIG. 3. The treatment(s) received and outcome(s) for each of the patient's in the data set is known. For example, if a patient received an immunotherapy treatment (e.g., anti-PD-1 immunotherapy), it is known whether such immunotherapy treatment was successful (disease was responsive) or unsuccessful (disease was non-responsive). Additionally, the data set can include patient data for one or more patients before administration of immunotherapy and after administration of immunotherapy. For each of the patients in the data set, the respective patient data can be clustered and violin plots can be generated as described herein. Accordingly, the data set contains information about which patients are receptive to immunotherapy treatment, as well as which immune cell phenotypes and/or nodes are relevant to such responsiveness.

When patient data for the cancer patient (i.e., a new patient who has not yet received immunotherapy or targeted therapy treatment) is received, clustered, and violin plots generated (e.g., FIG. 1, Steps 102, 104, and 106), the data set can be referenced to predict whether the cancer patient will respond to immune-based therapy. For example, the statistical analysis of the violin plots can be used to detect a variation (e.g., even a subtle variation) in immune cell phenotypes within a node between the cancer patient (i.e., the cancer patient whose data is received at FIG. 1, Step 102) and the patients whose patient data is contained in the data set. This disclosure contemplates that such variation(s) can be used to make a prediction. Alternatively or additionally, the statistical analysis of the violin plots can be used to determine which of the nodes of the clustered patient data are associated with response to immune-based therapy. In addition to the traditional markers, the size and/or physical parameters of cells such as but not limited to scatter properties of the cells (Side Scatter (SSC) and Forward Scatter) are utilized to further cluster the cells. Alternatively or additionally, the statistical analysis of the violin plots can be used to determine which of the nodes of the clustered patient data are associated with non-response to immune-based therapy. In other words, the nodes may be used as a biomarker or signature. The statistical analysis can be at least one of a principal component analysis, a cluster analysis technique, a distance matrix analysis, a Cox regression analysis, or a Wilcoxon signed-rank test. In the case of image microscopy data, statistics on the distance matrix between cell types (in addition to the phenotypes themselves) can also be utilized in the same regard to determine response to therapy. It should be understood that these statistical analyses are provided only as examples and that others can be used.

As described below, in some implementations, immune cell phenotype dimension reduction can be performed after generating the violin plots (e.g., FIG. 1, Step 106) and before performing the statistical analysis (e.g., FIG. 1, Step 108). The immune cell phenotype dimension reduction is to take the number of cells in each node as the variable and associate it with response. In this implementation, the algorithm does not utilize the distribution of the events within the violin plot for statistical analysis. In addition to the traditional markers, the size and/or physical parameters of cells such as but not limited to scatter properties of the cells (Side Scatter (SSC) and Forward Scatter) are utilized to further cluster the cells. Instead, the markers facilitate clustering of the cells, but ultimately it is the number of events in each node in responder versus non-responder that is used by the algorithm. For example, the statistical analysis of the number of events within a node can be used to detect a variation in immune cell phenotypes within a node between the cancer patient (i.e., the cancer patient whose data is received at FIG. 1, Step 102) and the patients whose patient data is contained in the data set. This disclosure contemplates that such variation(s) can be used to make a prediction. Alternatively or additionally, the statistical analysis of the number of events (e.g. cells) within a node (phenotype) can be used to determine which of the nodes of the clustered patient data are associated with response to immune-based therapy. Alternatively or additionally, the statistical analysis of the number of events within the node can be used to determine which of the nodes of the clustered patient data are associated with non-response to immune-based therapy. In other words, the number of events in each of the nodes may be used as a biomarker or signature. The statistical analysis can be at least one of a principal component analysis, a cluster analysis technique, a distance matrix analysis, a Cox regression analysis, or a Wilcoxon signed-rank test. In the case of image microscopy data, statistics on the distance matrix between cell types (in addition to the phenotypes themselves) can also be utilized in the same regard to determine response to therapy. It should be understood that these statistical analyses are provided only as examples and that others can be used.

Optionally, the method can include recommending an immunotherapy (e.g., anti-CTLA4 immunotherapy or anti-PD-1 immunotherapy) for the cancer patient that is predicted to respond to immune-based therapy. This disclosure contemplates that the recommendation can be performed using a computing device such as computing device 300 shown in FIG. 3. Optionally, the method can further include administering an immunotherapy (e.g., anti-CTLA4 immunotherapy or anti-PD-1 immunotherapy) to the cancer patient that is predicted to respond to immune-based therapy. It should be understood that anti-CTLA4 immunotherapy or anti-PD-1 immunotherapy are provided only as an example and that other immune-based therapies can be recommended and/or administered. This disclosure contemplates recommending and/or administering any known immunotherapy. One example immunotherapy drug is pembrolizumab such as KEYTRUDA from MERCK & CO., INC. of Kenilworth, N.J. Optionally, the new patient data for the patient who receives immunotherapy (including whether or not the new patient responds thereto) can be added to the data set.

Alternatively or additionally, the method can optionally include performing immune cell phenotype dimension reduction. It should be understood that each node of the clustered patient data has a certain number of cells with a particular phenotype. The immune cell phenotype dimension reduction includes taking the number of cells (events) in each node for each particular patient to the statistical analysis rather than try to analyze each antibody target individually. As described herein, phenotype dimension reduction can be performed after generating the violin plots (e.g., FIG. 1, Step 106) and before performing the statistical analysis (e.g., FIG. 1, Step 108). FIG. 2D illustrates an example schema for immune cell phenotype dimension reduction. Phenotype dimension reduction can be used to reduce the number of events. By reducing the number of events, it is possible to reduce the burden on the computing device's resources.

FIG. 2A illustrates clustering of immune cell phenotypes. Seven myeloid (HLADR, CD33, CD11b, CD14, CD15, CD11c, CD103, DAFFM-NO stain) and nine lymphoid (CD3, CD4, CD8, CD56, CD25, CD127, CD19, CD11c, DAFFM) were used to characterize cellular subsets derived from patients undergoing adjuvant anti-CTLA-4 therapy based on the fluorescence intensity. Cells were stained, fixed in 1% formaldehyde, and analyzed on an LSR II flow cytometer (100,000 live events) using standard gates, isotype control antibodies and compensation beads to establish criteria for positive staining and compensation controls. The data was visualized using the MPAT-R application, and the FCS Express software (Glendale, Calif.). For the anti-PD-1 studies, the CD103 molecule that distinguishes BAFT3 DCS can also be used. The SPADE clustering algorithm was used for FIG. 2A. FIGS. 2B and 2C illustrate violin plots. The violin plots were constructed with positive/negative cut-offs for each of the markers. The application can display the violin plots for each node (or cluster), which is shown in FIG. 2B, or for each sample, which is shown in FIG. 2C. In addition, the application can scale the violin plots to the number of events in the node/sample. Each row is labelled by the node/sample number and the number of events in that node/sample. FIG. 2D illustrates phenotype dimension reduction, i.e., the schema for the number of events per node versus patient sample. FIG. 2E is a flow chart illustrating example operations for the MPAT-R algorithm. After the phenotype dimension reduction where an eight parameter flow cytometry stain is reduced to the number of events in a node for a particular sample, statistics can be utilize to determine which nodes are associated with the best or worse response to therapy. The visualization tool can assist to find the populations in conventional flow cytometry software for further visualization.

Example Computing Device

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which embodiments of the invention may be implemented is illustrated. It should be understood that the example computing device 300 is only one example of a suitable computing environment upon which embodiments of the invention may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

### Example 1

As described above, a blood sample contains millions of immune cells, and each cell can be described based on its expression of immune markers. This results in many thousands of combinations of immune cell phenotypes. The SPADE (spanning-tree progression analysis of density-normalized events) algorithm can be used to accurately cluster immune cell phenotypes. The SPADE algorithm represents all the cells in the experiment and displays potential immune cell sub-types into a tree (see FIG. 2A). The SPADE algorithm differentiates cell populations based upon a particular marker. The trees showing different types of cells containing one specific marker (nitric oxide) are shown. Each circle represents a proportion of the total number of cells with the levels of nitric oxide in a color scale of blue (negative) and red (highest intensity).

Phenotypic identification to differentiate responding patients' trees from non-responding patients is a laborious process once the clustering of the cell phenotypes is complete. Therefore, an algorithm, MPAT-R (multi-parameter phenotyping analysis tool in R) has been developed to quickly determine the cell phenotypes (see FIGS. 2B and 2C). FIG. 2C illustrates violin plots for a plurality of samples (e.g., patient blood samples). Each column represents a flow cytometry marker (Myeloid Markers (left) and Lymphoid Markers (right)), and each row represents a particular sample. A violin plot describes the signal intensity of the fluorophore and captures the number of events to facilitate a statistical analysis (e.g., principal component analysis). In addition, the same analysis can be visualized on a per node basis as shown in FIG. 2B. Utilizing this technique, subtle variations in immune cell phenotypes within a node between patients can be detected.

The disclosure contemplates providing a software application in one example implementation. The software application can allow clinicians to quickly determine which immune cells are important for response to therapy in the clinical trial setting. Most clinical trials do not employ high dimensional analysis for immune cell markers due to the lack of the ability to analyze these data sets in a meaningful way. This software package can be broadly applicable to flow cytometry and other similar techniques and can bridge the gap between algorithms to sort cell phenotypes and the needs of the clinic. The software application can identify those patients that will respond to therapy, avoid unnecessary toxicity in patients who are unlikely to respond, and to further the understanding of the biology of immune checkpoint blockade to facilitate the next generation of immune based therapeutics. There are also promising results that show that it is possible to ‘prime’ a patient's immune system, making the patient more likely to respond to therapy, but all of this requires accurately and easily describing the immune system.

To develop the MPAT-R algorithm and to test whether nitric oxide is differentially expressed in different immune cells, two flow cytometry panels (100,000 live cell events collected on a LSR II flow cytometer) were constructed to compare immune cells collected pre and post ipilimumab/vaccine treatment (e.g., an example immunotherapy treatment) (Myeloid panel: nitric oxide stain (DAFFM), HLADR, CD33, CD11b, CD14, CD15, and CD11c; Lymphoid panel: DAFFM, CD3, CD4, CD8, CD25, CD127, CD56, CD19, and CD11c). Controls included utilization of flow cytometric compensation beads to establish robust compensation matrices, fluorescence minus one controls to set negative and positive gates, isotype controls to control for patient variations, and a live/dead marker. Patients with resected stage III/IV melanoma were treated with ipilimumab plus a peptide vaccine, and pre and post treatment peripheral blood mononuclear cells (PBMCs) were available for analyses. Example operations performed by the software application are described below.

Step 1: Upload the live patient cells from the flow cytometry data set. These files can be prepared in a batch format utilizing a flow cytometry visualization program (e.g. FCS Express 6).

Step 2: The program can run the SPADE algorithm (see FIG. 2A).

Step 3: Determine the positive/negative cut-off for each parameter. In some implementations, the violin plots were displayed and the user can visually (manually) define the positive/negative cut-points. In other implementations, the negative flow cytometry control files can be read and the cut-off for positive/negative can be automatically determined for that particular parameter.

Step 4: Display the violin plots (FIGS. 2B and 2C) of the markers' signal intensity across all the nodes including the cut-offs for negative/positive. This step allows the user to visually check whether a clinically meaningful number of nodes (potential cell types) were chosen. In some implementations, it may be possible to computationally determine the most suitable number of immune cell subsets (nodes) present in any particular sample set. Improving the efficiency of the algorithm can be accomplished by making use of parallel processes to facilitate use of all the cores and random access memory (RAM) available on the computer dedicated to this task.

Step 5: Superimpose new flow cytometry samples on the tree. The SPADE algorithm can be used to down-sample the data. For example, instead of creating a brand new tree, the down-sampled data of the current specimen can be added to the known tree. A measure of fitness can be the percentage of cells that do not fit into currently available nodes.

By the MPAT-R pilot algorithm and subsequent statistical analysis, many lymphoid and myeloid phenotypes were differentially expressed after ipilimumab/vaccine treatment (Wilcoxon signed-rank test) as shown in the tables. This rich data set can be used to further develop the algorithm. Debatching methods and additional statistical analyses can be performed to ensure that batch experimental effects have minimal effect on determining biological outcomes. Permutation of the responder status can be performed to generate the empirical null distribution to account for potential inter-dependency of the nodes in the SPADE tree.

### Example 2

The immune system has a role in the development, progression and effective treatment of melanoma. Blockade of inhibitory immune pathways such as CTLA-4 and PD-1 with neutralizing monoclonal antibodies has been shown to lead to regression of disease. The response rate of single agent anti-PD-1 is approximately 30-40%, and in combination with anti-CTLA-4 therapy response rates increase to 50-60%, at the cost of significantly increased toxicity. However, many patients do not benefit from these therapies.

Cancer cells (such as, for example, melanoma cells) are recognized by the immune system and can be killed by T lymphocytes and natural killer (NK cells), but the anti-tumor activity of T and NK cells is abrogated by tumor-mediated mechanisms including depletion of nutrients from the tumor microenvironment, production of reactive oxygen and nitrogen species, secretion of immune-suppressive cytokines, and induction of inhibitory immune cells. Presentation of antigens to T cells by dendritic cells (DCs) is defective in the setting of melanoma. Recently, it has been shown that stimulation of DCs with type I interferons (IFN-α and β) and down-stream signal transduction via the janus kinase-signal transducer and activator of transcription (Jak-STAT) pathway are critically important to immune surveillance and the generation of effective host T cell immune responses to cancer. It is already known that derangements of the JAK-STAT pathway have been implicated in some cases of anti-PD-1 resistance (mutations in the JAK2 protein), but it is likely that functional derangements in STAT1 are more pervasive (and more subject to intervention) than mutations in JAK2. Infiltration of immune cells obtained from post treatment biopsies are associated with response but are not of sufficient sensitivity or specificity to use in the clinical setting. Furthermore, in DCs, IFN-α signaling is responsible for up-regulation of class I and class II MHC molecules for the presentation of antigens on DCs. In particular, increased MHC-II expression levels in melanomas are associated with response to anti-PD-1 therapy. It has been demonstrated that the anti-tumor effects of IFN-α were dependent on STAT1 signal transduction in immune cells via phosphorylation of tyrosine 701 and JAK-STAT signaling was markedly inhibited in human peripheral blood immune cells from tumor bearing patients. More recently, a mechanism of immune inhibition was elucidated that involves secretion of NO by tumor-induced inhibitory immune cells (known as myeloid-derived suppressor cells, MDSC) and subsequent decreased p-STAT1 signaling in response to interferon signaling. It has been demonstrated that NO inhibits antigen presentation from DCs to T cells and that STAT1 is nitrated at position 701 in human melanoma samples. Production of NO by MDSCs leads to the production of reactive nitrogen species which are chemical entities inside cells derived from NO that cause nitration at key amino acids such as tyrosine. MDSCs arise from myeloid precursors in response to tumor-derived growth factors and pro-inflammatory cytokines (e.g., IL-6, GM-CSF). Their presence is correlated with tumor burden and is predictive of low overall survival. In humans, MDSCs are described by both their functional capacity to suppress T cell activation and immature myeloid phenotype (typically CD33⁺CD11b⁺HLADR^{low/-}). MDSC number increases in patients with poor response to anti-CTLA-4 treatment, and the level of NO increases with more advanced stages of melanoma. Earlier work has demonstrated that there is an increased MDSC population associated with poor anti-PD-1 response in melanoma. Given the discovery that MDSCs nitrate STAT1 and the importance of DC Jak-STAT signaling in the generation of an effective host immune response, MDSC-mediated nitration of STAT1 in DCs and T cells may be an important mechanism of immune inhibition in the setting of melanoma and reversal of this inhibition will may to improved anti-tumor immunity in the setting of anti-PD-1 therapy.

Levels of NO produced by immune cell suppressor cell subsets in PBMCs from melanoma patients can be measured before and after treatment with anti-PD-1 and correlated with changes in immune cell responses to interferon levels. NO levels can be examined in immune cell subsets in PBMC samples and analyzed using the MPAT-R algorithm described herein (see FIGS. 2A-2E). MDSCs within the peripheral blood of melanoma patients can be phenotyped (see FIGS. 2A-2E). Although MDSCs are believed to be the major contributor of NO, T, NK, B, DC immune subsets and normal donor PBMCs can also be characterized to determine their NO content as well. An aliquot of PBMCs from the patients can be stimulated in parallel with IFN-α and subsequent downstream activation can be determined via measurement of phosphorylated STAT1. Activation of STAT1 can be measured by intracellular staining for phosphorylated STAT1 using flow cytometry.

The blood samples can be collected from patients at two time points (e.g., immediately before treatment initiation on day 0 and prior to the second dose of therapy and two to three weeks after the first dose of anti-PD-1 generally, immediately before the patient receives their second dose of therapy). The following information can be collected 1) objective response (RECIST and immune-related response criteria); 2) complete response rate; 3) “clinical benefit rate” (CR+PR+SD for at least 24 weeks); 4) progression free survival time; 5) % progression free at 6 and 12 months; 6) duration of objective response from all patients and conduct exploratory analyses to see which endpoint(s) best correlate with immune dysfunction. The change of NO+MDSC levels between baseline and prior to the second dose of anti-PD-1 therapy can be compared using a one-sample t-test. The differences observed in NO⁺MDSC level change between progressors and non-progressors, and their variabilities can be estimated. In addition, the flow cytometry data from anti-PD-1 can be superimposed on the 200 known phenotypes for the anti-CTLA-4 dataset as well as generating new phenotypic trees using MPAT-R. Combat, a debatching method, can be performed to ensure that potential batch experimental effects can be removed before association will be performed. Cox regression models can be performed to estimate the potential effect size for the association between progression free survival and the number of events pre and/or post treatment in other cell populations (node in MPAT-R analysis). Permutation of the responder status can be performed to generate the empirical null distribution to account for potential inter-dependency of the nodes in the SPADE tree. The assay can be used to identify patients who will not gain at least one year of disease control with single agent-PD-1, and such patients can be considered candidates for more aggressive treatment with the combination anti-CTLA-4/anti-PD-1 despite its much higher toxicity and also appropriate for clinical trials of anti-PD-1 plus anti-NO agents with a study design aimed at significantly increasing the percentage of patients who achieve 12 month PFS.

Bioinformatics/Biostatistics methods for analyzing array based data (miRNA, chemokine arrays) can assess involvement of NO and IFN dependent processes. NO-dependent processes can upregulate miRNAs (e.g. miRNA-21) that are known to be upregulated in melanoma. In the case of miRNA-21 it is known that it suppresses expression of chemokines such as IP-10. Therefore, it is reasonable to suspect that specific miRNAs are responsible for differential chemokine expression that can then alter the processes related to antigen presentation and the ability of the host immune system to appropriately respond to cancers such as melanoma. A signature that correlates with the levels of nitration of the STAT1 protein can be discerned, and in doing so more insight into the mechanism of how NO dependent pathways inhibit antigen presentation from DC to T cells can be gleaned. Four pre/post anti-PD-1 (PBMC, plasma) and 30 tissue samples prior to anti-PD-1 therapy were run on an HTG EdgeSeq Processor (HTG Molecular Diagnostics, Tucson, Ariz.) using the HTG EdgeSeq miRNA whole transcriptome (WT) and PBMC samples can be run on immuno-oncology assays (549 RNA transcripts of genes involved in the immune response to cancer including chemokines and checkpoint receptors). The initial bioinformatics biostatistical analysis can be similar to a recent publication analyzing miRNA profiles from melanoma patients. A Bayesian graphical modeling approach can be utilized to associate levels of miRNA and chemokine expression. A similar process can be utilized for both shotgun proteomics experiments conducted on 20/30 tissue samples listed above. This process can produce a list of potential miRNAs/chemokines that will be interrogated by bioinformatics tools such as GeneGo, Pantherdb, Enrichr, and GSEA to assess whether the NO and interferon pathways are involved. The output from the miRNA and chemokine data sets can be directly associated with the immune cell populations and levels of NO obtained from the MPAT-R analysis. A chemokine or miRNA profile with a particular immune cell subset may be found to build a model to predict response to immune checkpoint blockade in melanoma patients.

### Example 3

Phenotyping of immune cell subsets in clinical trials is limited to a few well-defined phenotypes, due to the technological limitations of reporting novel flow cytometry multi-dimensional phenotyping data. A multi-dimensional phenotyping analysis tool (MPAT-R) has been developed and applied to the detection of nitric oxide levels in peripheral blood immune cells before and after adjuvant ipilimumab co-administration with a peptide vaccine in melanoma patients. The algorithm (MPAT-R) allows for immune cell phenotypes to be visually inspected without knowledge of clustering techniques and to categorize nodes by association to relapse-free survival. Using the analysis approach described herein, nitric oxide was found in the immune-stimulatory effector cells obtained from patients with longer-term (>1 year) relapse-free survival and in immune-suppressor cell subsets associated with shorter-term (≤1 year) relapse-free survival, arguing for a dichotomous role of nitric oxide in pro- and anti-tumor effects. Phenotyping of immune cells using this tool is not limited specifically to nitric oxide phenotyping, and it can now be applied in the monitoring of anti-tumor effects of a variety of immunotherapeutics in cancer patients.

Monoclonal antibodies against cytotoxic T-lymphocyte-associated antigen 4 (CTLA-4), ipilimumab, and programmed cell death protein 1 (PD-1), pembrolizumab and nivolumab, are approved for patients with advanced melanoma. However, response rates for ipilumumab and nivolumab in melanoma patients are 11% to 22% and 31% to 44%, respectively. Both types of checkpoint blockade are now Food and Drug Administration-approved for patients who have undergone surgery for metastatic melanoma, although it is unclear which patients require upfront therapy and which would benefit from waiting to start checkpoint blockade until the melanoma is visible on traditional imaging. Therefore, tools are needed to assess how effective the therapies are likely to be in different clinical scenarios.

Peripheral blood contains many types of immune cells that can be monitored and demonstrate change with therapy. High-dimensional flow cytometry phenotyping can be performed and analyzed via clustering algorithms including SPADE (Spanning-tree Progression Analysis of Density-normalized Events), t-SNE (t-Distributed Stochastic Neighbor Embedding), and viSNE (visualization tool for t-SNE). However, the outputs of these algorithms require manual curation based on marker expression for individual cells. To overcome this limitation, patient samples prior to and after adjuvant ipilimumab with a peptide vaccine treatment were phenotyped and a tool called the Multi-Dimensional Phenotyping Analysis Tool in R (MPAT-R) was developed to analyze associations between cell phenotypes and relapse-free survival (RFS). A multi-dimensional flow cytometry panel was developed to assess the algorithm and test the pro- and anti-tumor associations of nitric oxide (NO) levels in immune-suppressive or stimulatory peripheral blood immune cells. NO levels were measured in a broad range of immune cell subsets as NO and its metabolites have been shown to be elevated in immune suppressor cells derived from patients receiving anti-CTLA-4 therapy. While NO has traditionally been associated with immune-suppressive activity in clinical studies, the evidence for NO-meditated pro- and anti-tumor function via the activity of myeloid-derived suppressor cells (MDSCs), dendritic cells (DCs), cytotoxic T cells, and natural killer (NK) cells was recently described. The phenotyping tool described herein allowed for the analyses of high dimensional phenotyping data of NO in immune cells. This analysis is readily applicable to clinical trials by allowing for efficient unsupervised organization of distinct cell phenotypes.

Results

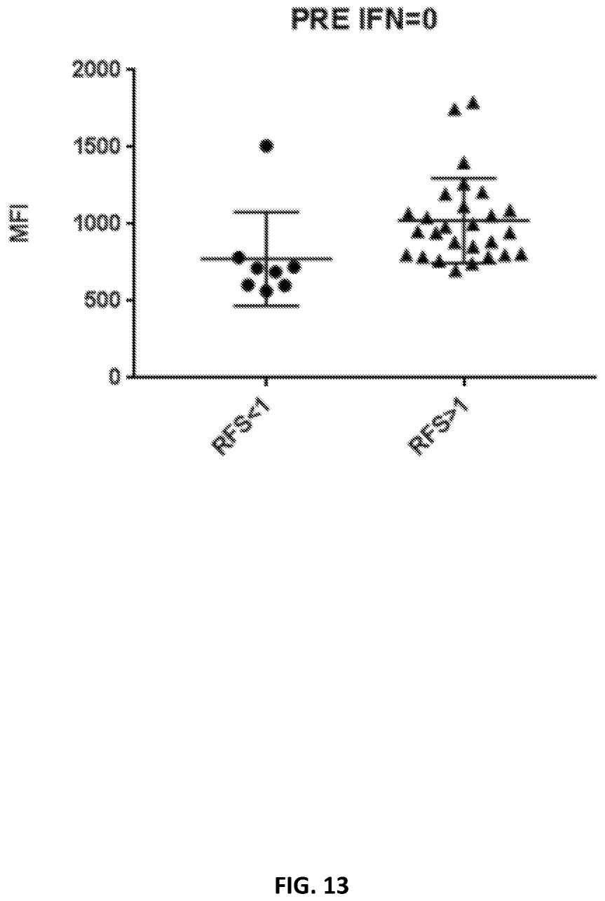

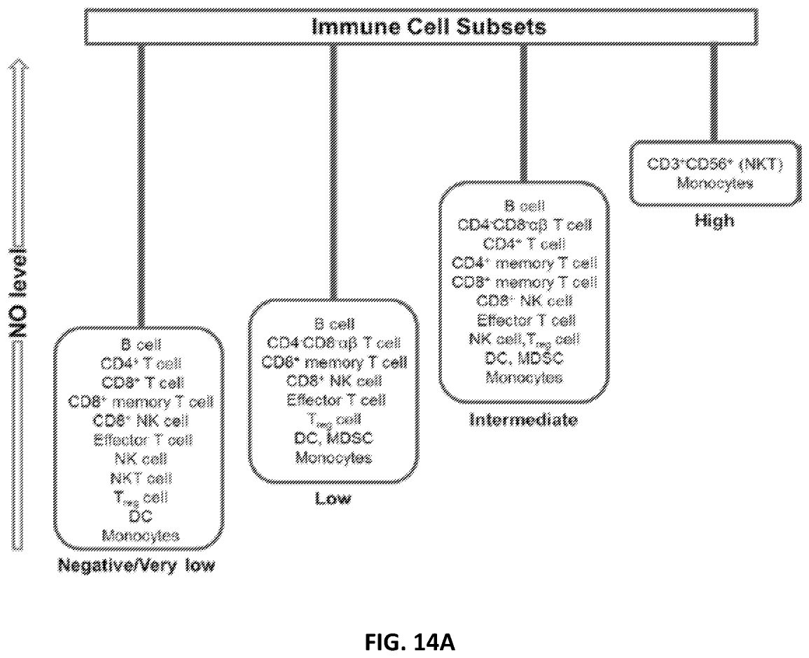

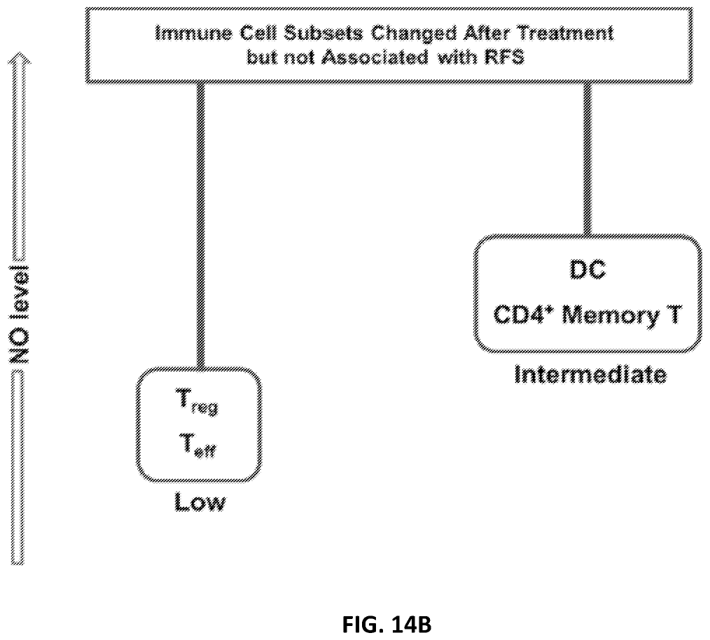

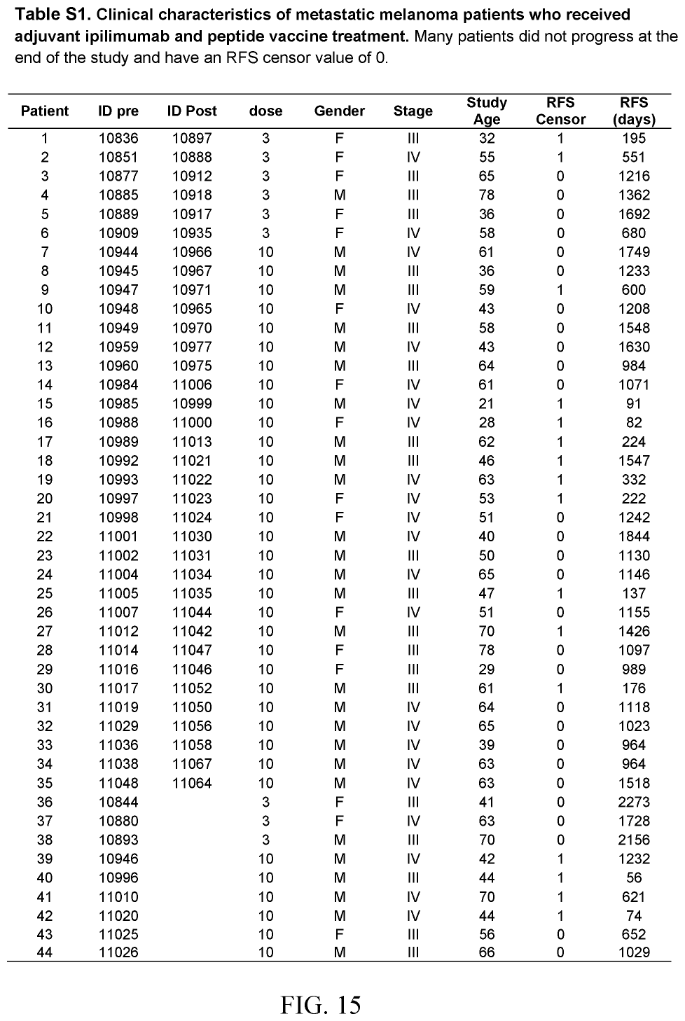

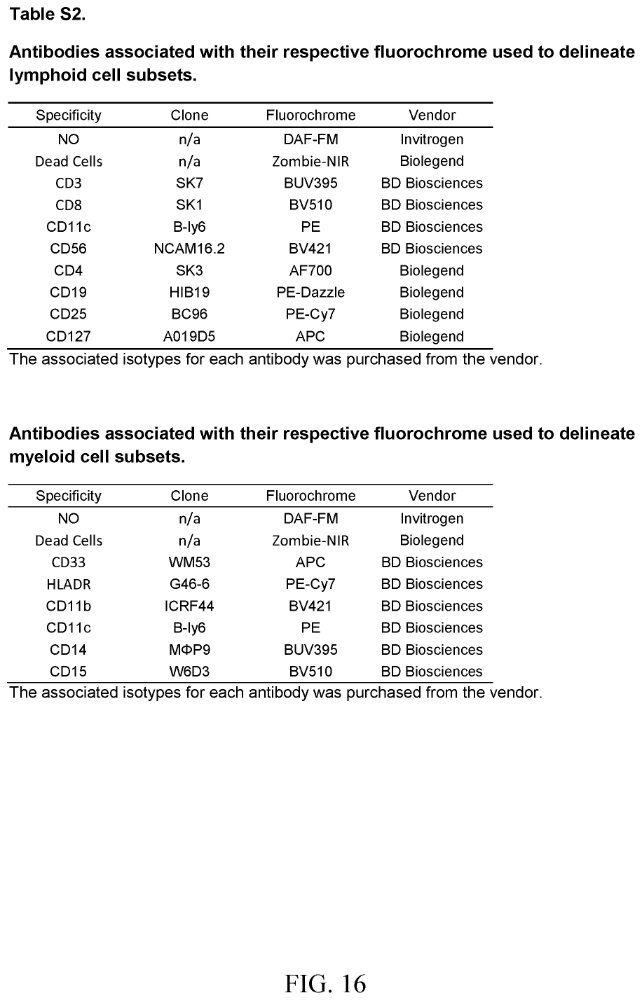

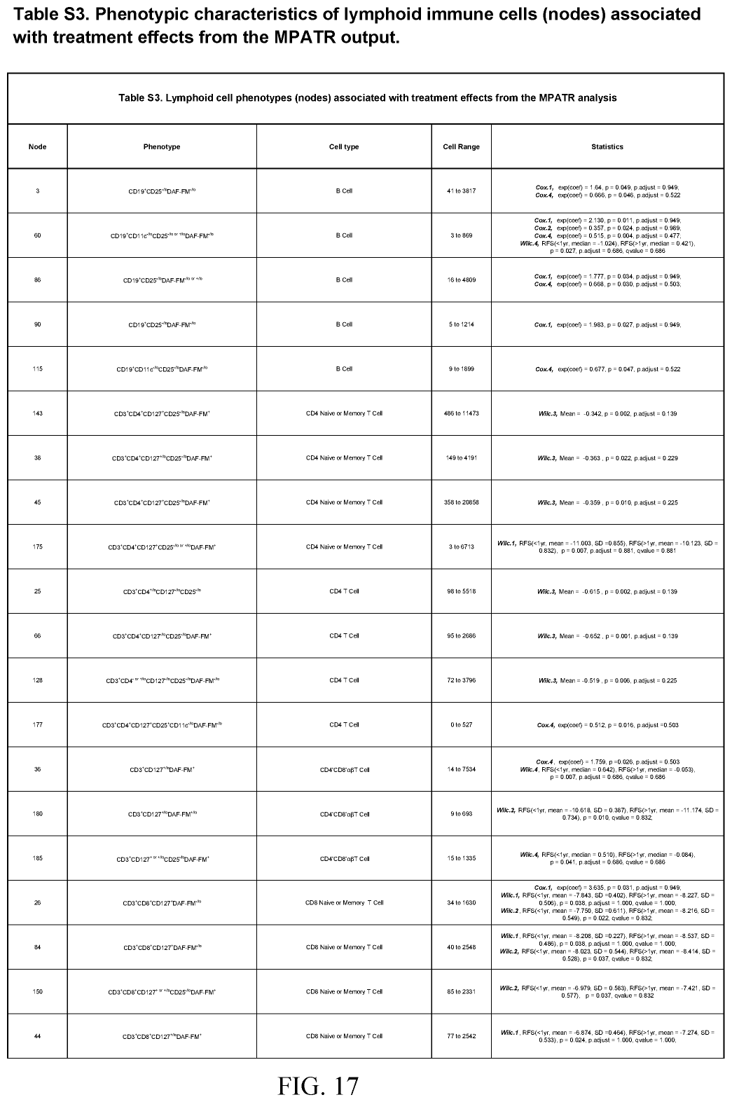

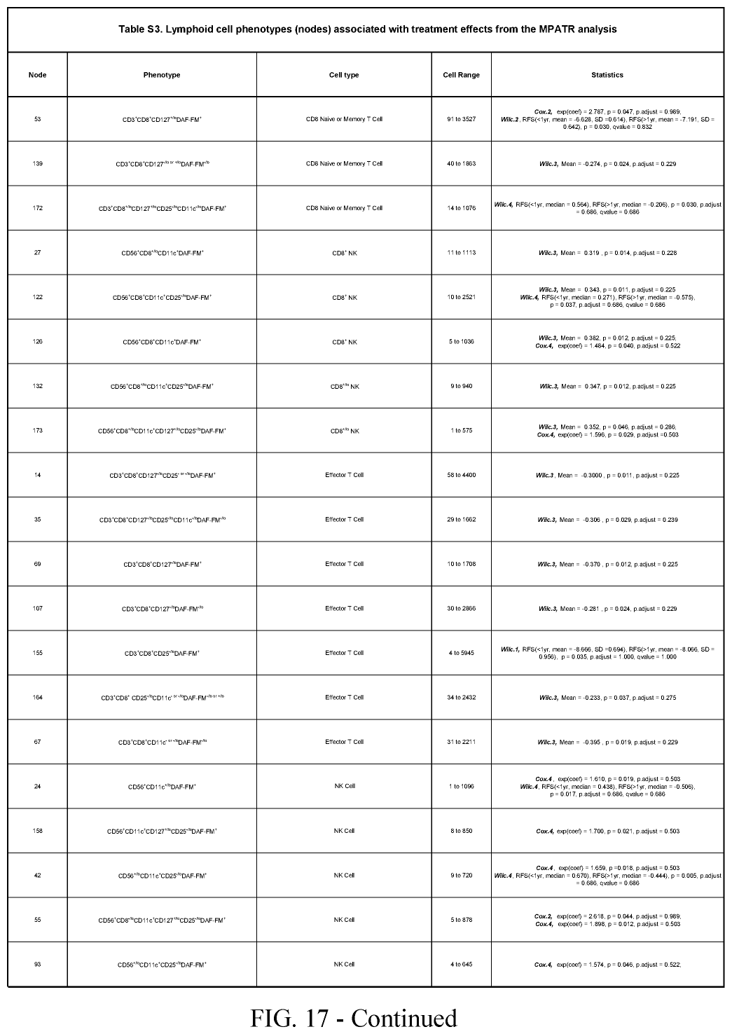

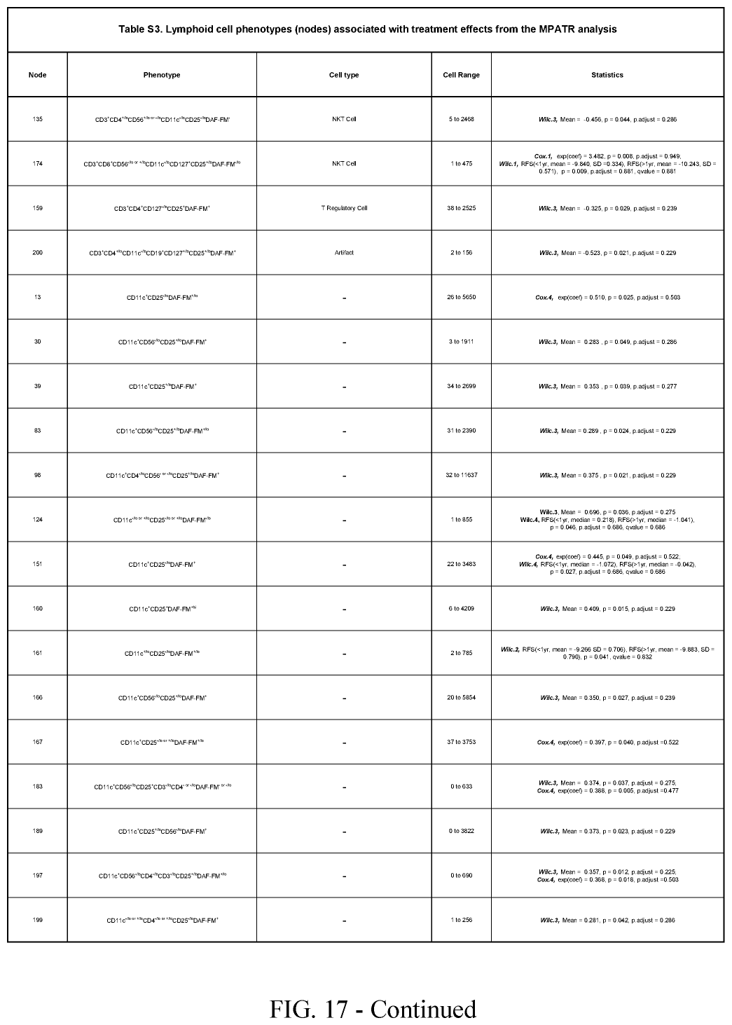

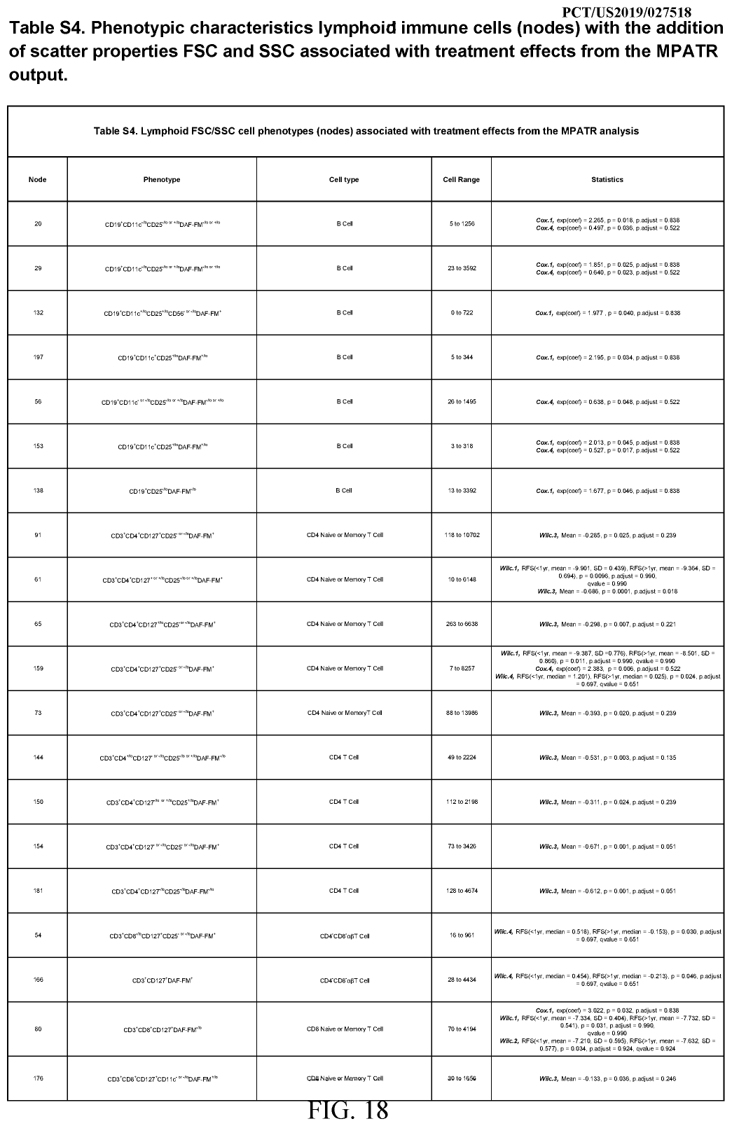

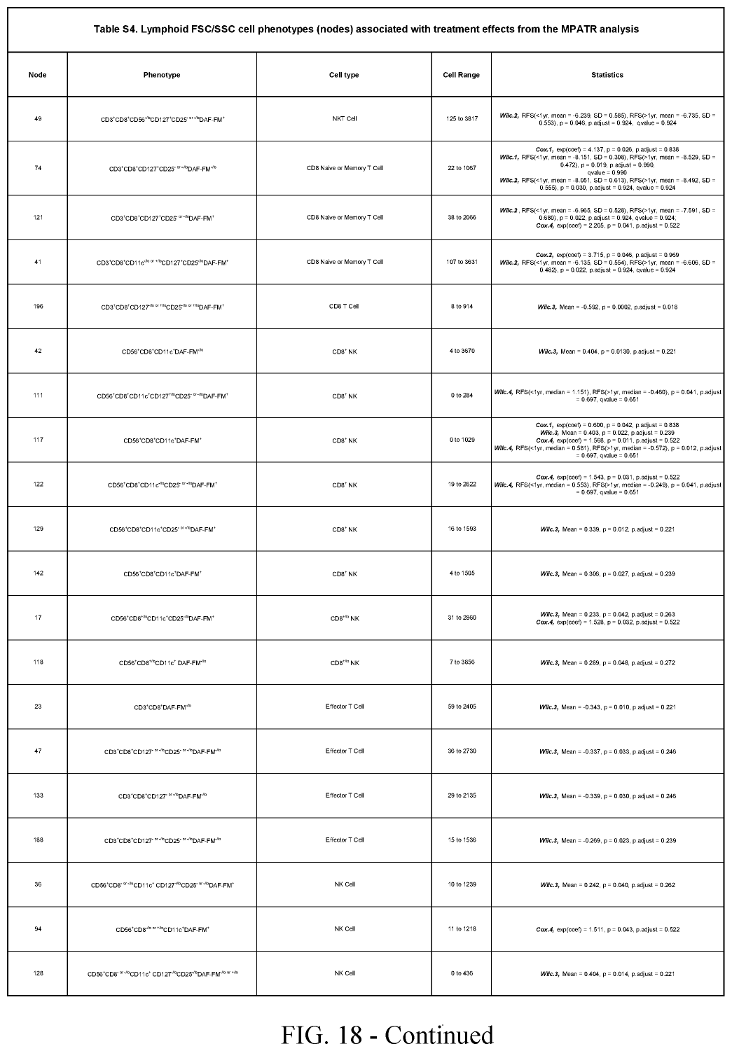

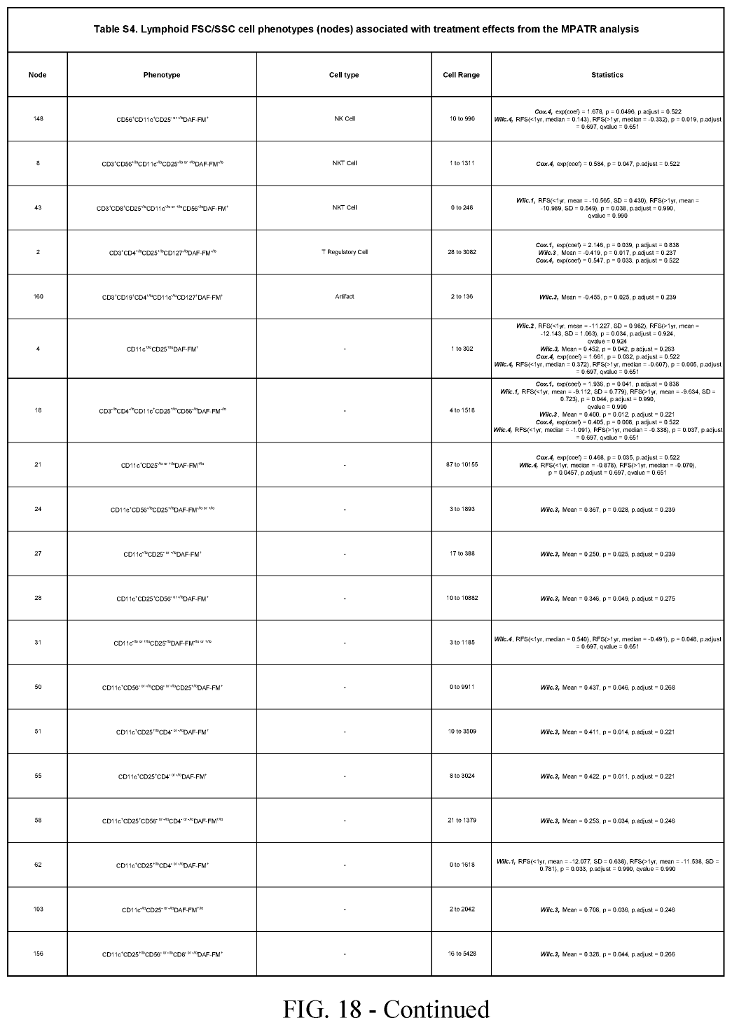

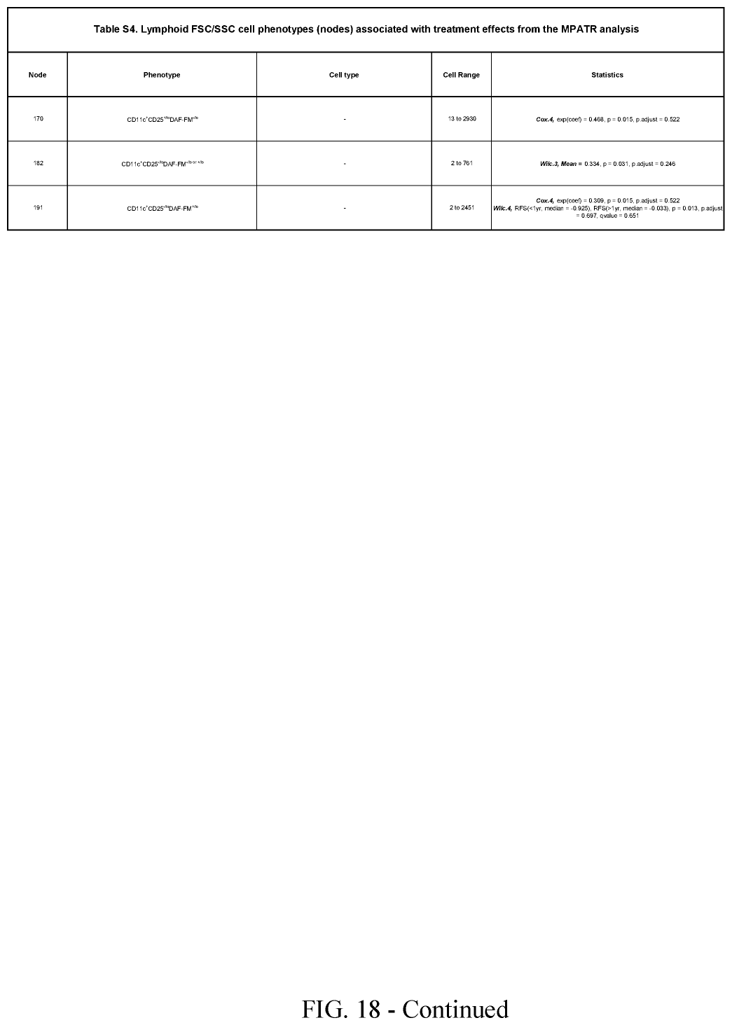

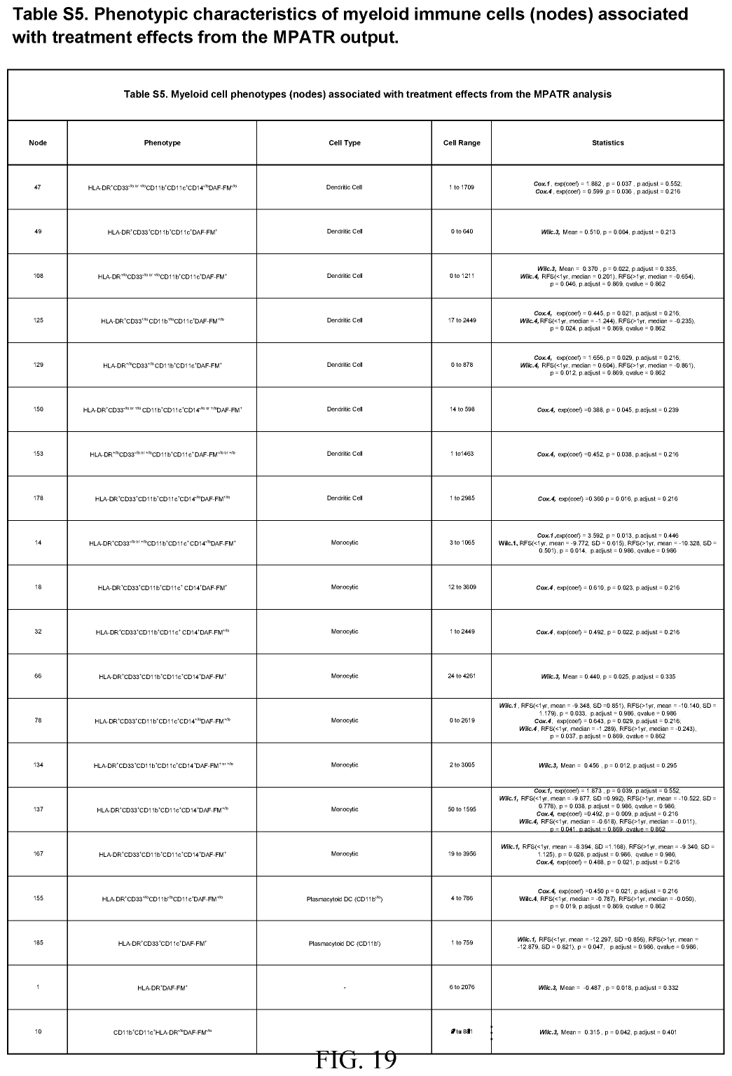

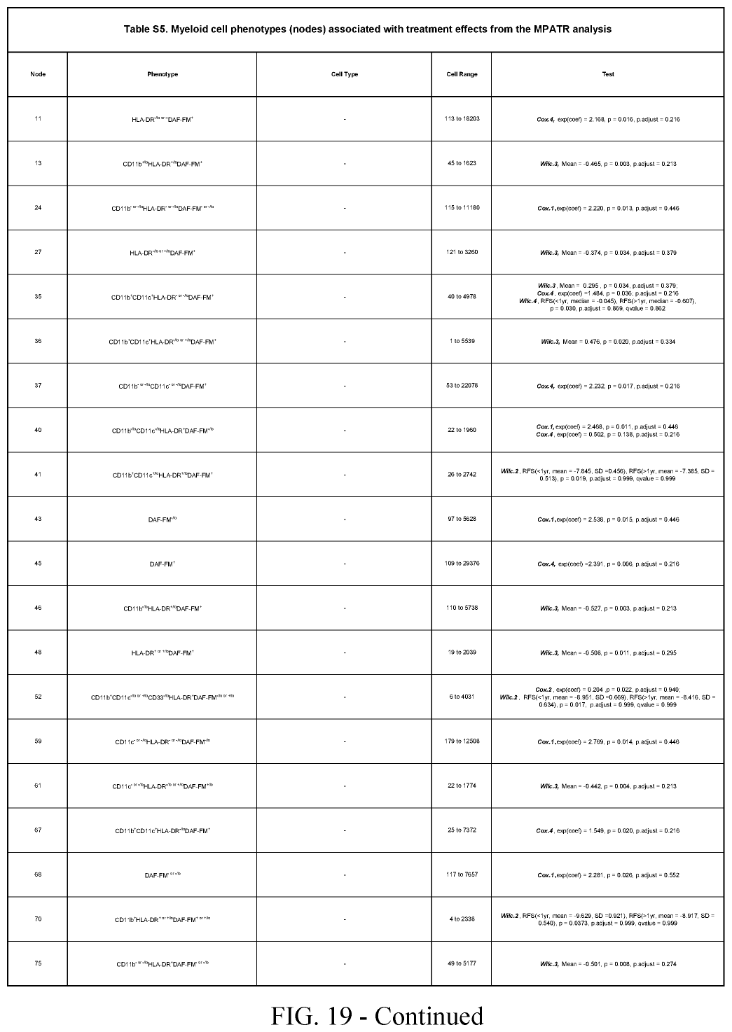

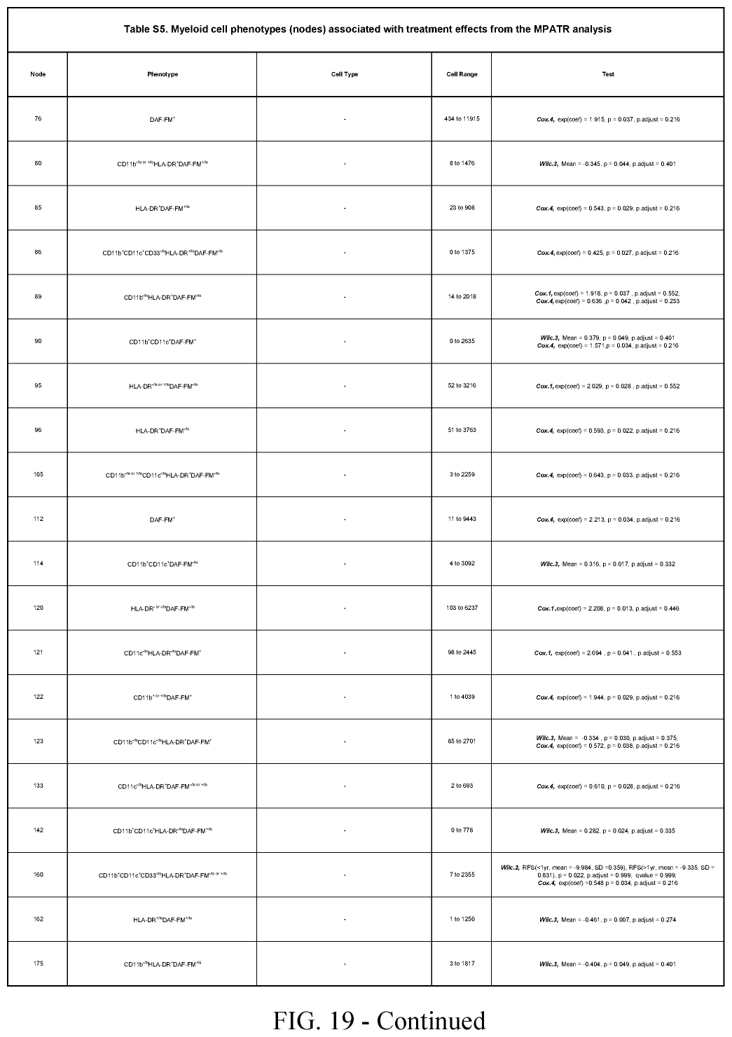

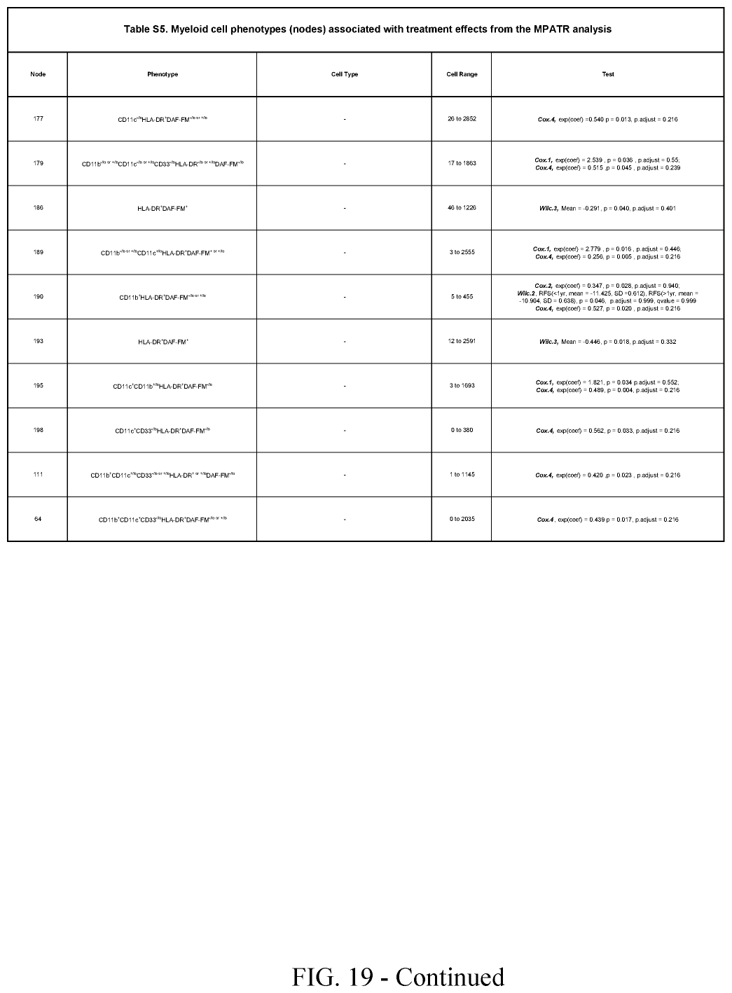

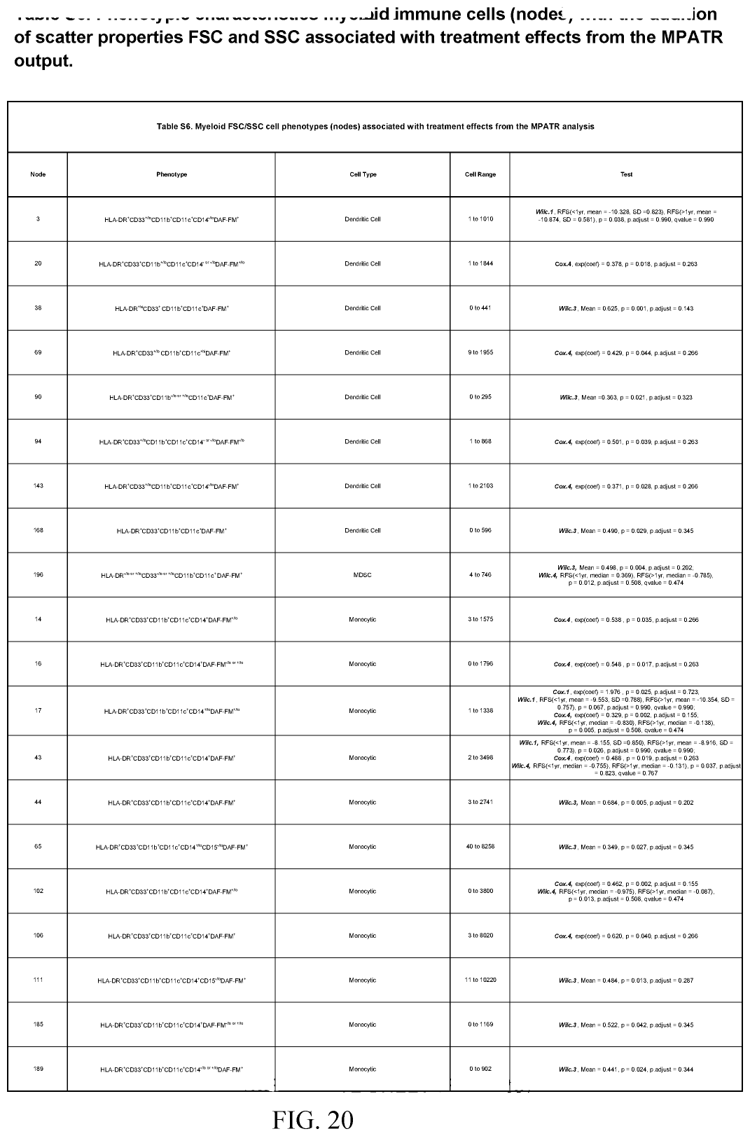

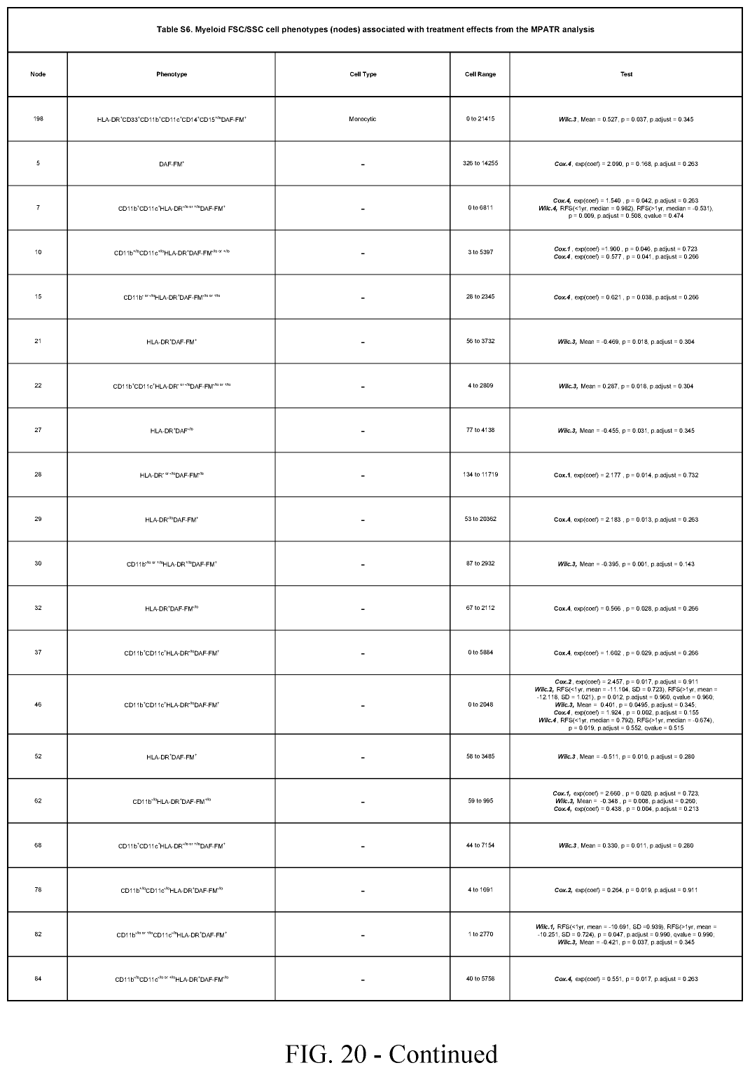

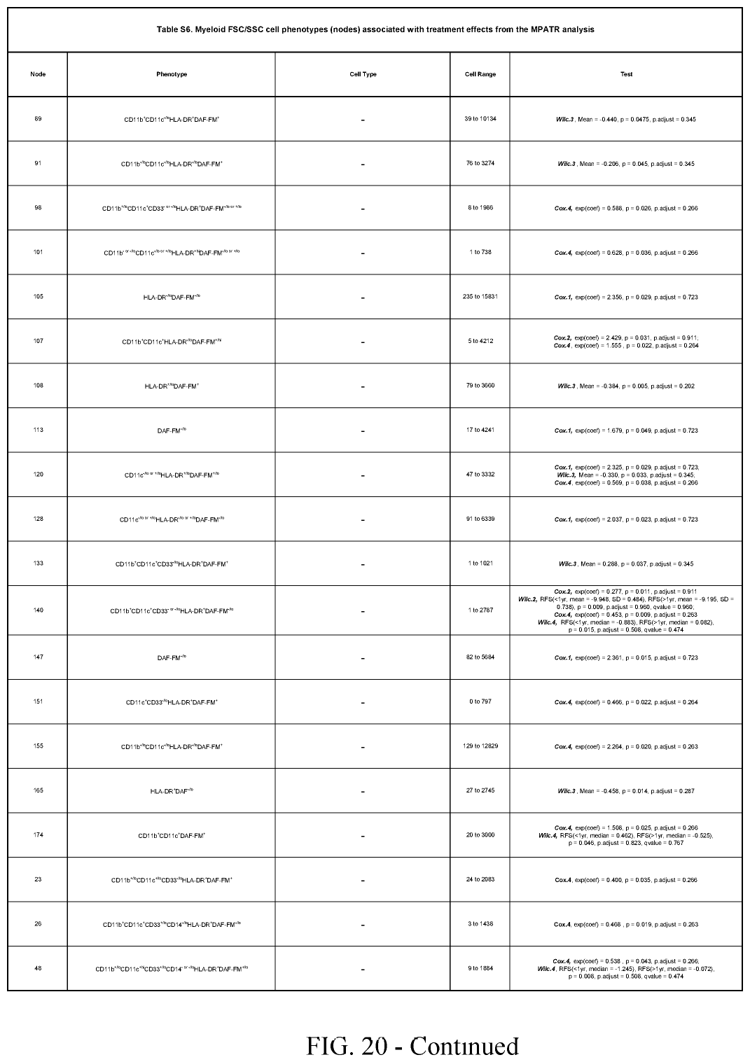

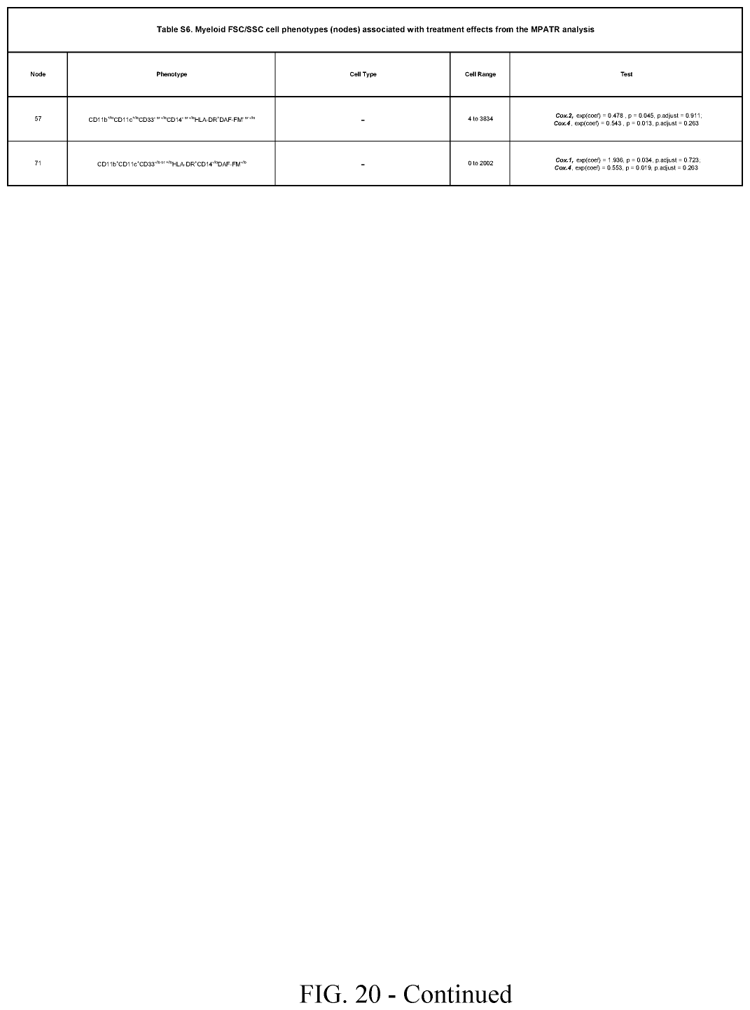

Patients with resected stage III/IV melanoma were treated with ipilimumab plus a peptide vaccine. Pre- and post-treatment peripheral blood mononuclear cells (PBMCs) drawn at week 13 of treatment were available for analysis (9 patients had pre-treatment samples only, 35 patients had both pre- and post-treatment samples; Table S1). As a control for this patient population, interferon response protein STAT1 phosphorylation levels were measured. As previously shown in the literature, pSTAT1 levels were higher in melanoma patients with longer-term RFS (FIG. 13, p=0.001, Wilcoxon rank sum test). High-dimensional flow cytometry analyses of patient PBMC samples were performed using lymphoid and myeloid panels that used DAF-FM as the NO stain (Myeloid panel: DAF-FM, HLA-DR, CD33, CD11b, CD14, CD15, and CD11c; Lymphoid panel: DAF-FM, CD3, CD4, CD8, CD25, CD127, CD56, CD19, and CD11c; Table S2). Cells were stained, fixed in 1% paraformaldehyde, and analyzed on an LSR II flow cytometer (100,000 live events) using standard gates, isotype control antibodies, and compensation beads to establish criteria for positive staining and compensation controls. The 488 nm Blue laser was reserved for DAF-FM, due to its extreme signal intensity, thus necessitating that the remaining antibodies use all other available lasers (405 nm Violet, 640 nm Red, 561 nm Yellow/Green, 355 nm Ultraviolet).

Development of MPAT-R Algorithm

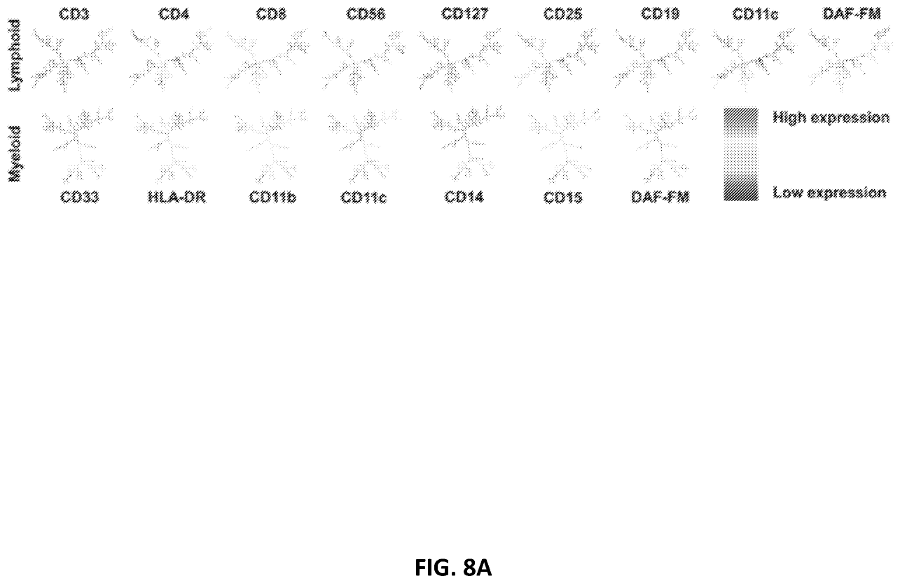

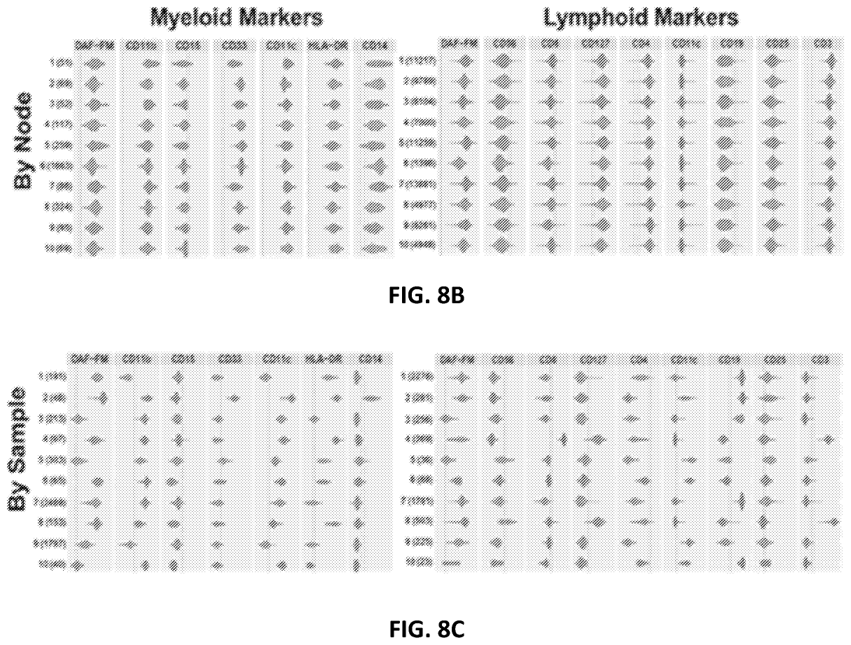

Referring now to FIGS. 8A-8C, the development and integration of the Multi-Dimensional Phenotype Analysis Tool in R (MPAT-R) algorithm, using patient samples is described. FIG. 8A shows nine lymphoid and 7 myeloid markers with and without the addition of scatter properties of cells (forward scatter area and side scatter area) from flow cytometry panels were used in the MPAT-R algorithm, to delineate the phenotypes of specific cell populations. In the first step, the different phenotypes of the cells were clustered using the SPADE algorithm, as shown by the SPADE trees. FIGS. 8B and 8C show the second step to visualize the clustering in a user-friendly way by using violin plots that were constructed with positive/negative cut-offs from a sample for each of the markers that were taken for visualization purposes. The MPAT-R application can display the violin plots for each node (cluster) or for each sample. In addition, the application can scale the violin plots to the number of events in the node/sample. Each row is labelled by the node/sample number and the number of events in that node/sample. The third step was to perform phenotype dimension reduction, for example as shown and described with respect to FIG. 2D. This process associates the number of events (cells) with each node (phenotype) to be used in downstream statistical analyses. After the phenotype dimension reduction, in which the multi-parameter flow cytometry stain is reduced to the number of events in a node for a particular sample, statistical analyses were performed to determine which nodes were associated with relapse-free survival, for example as shown and described with respect to FIG. 2E.

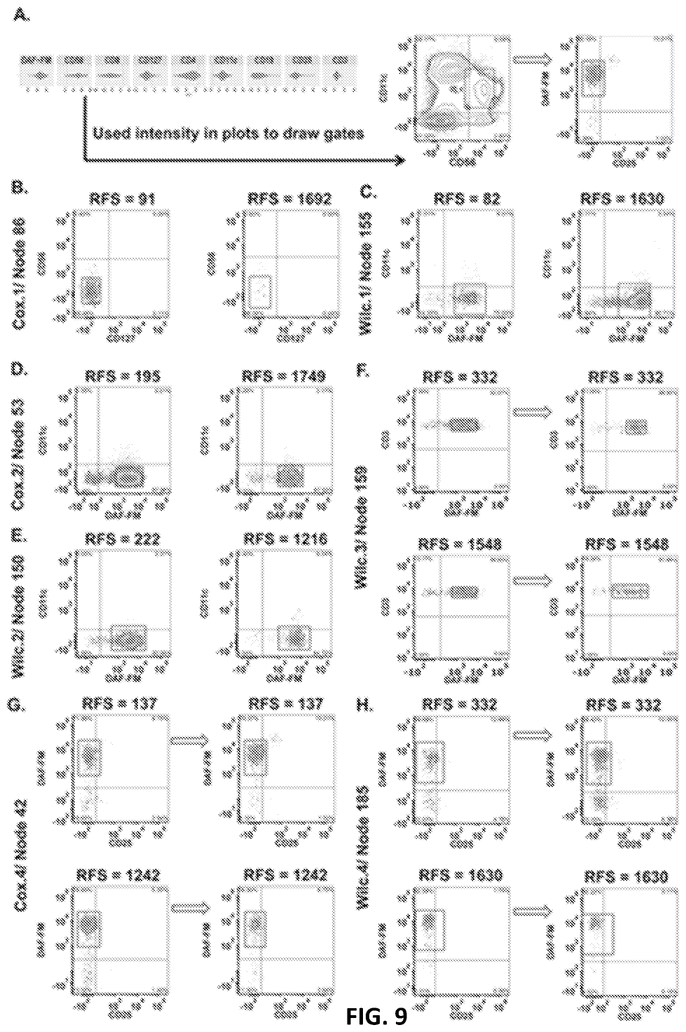

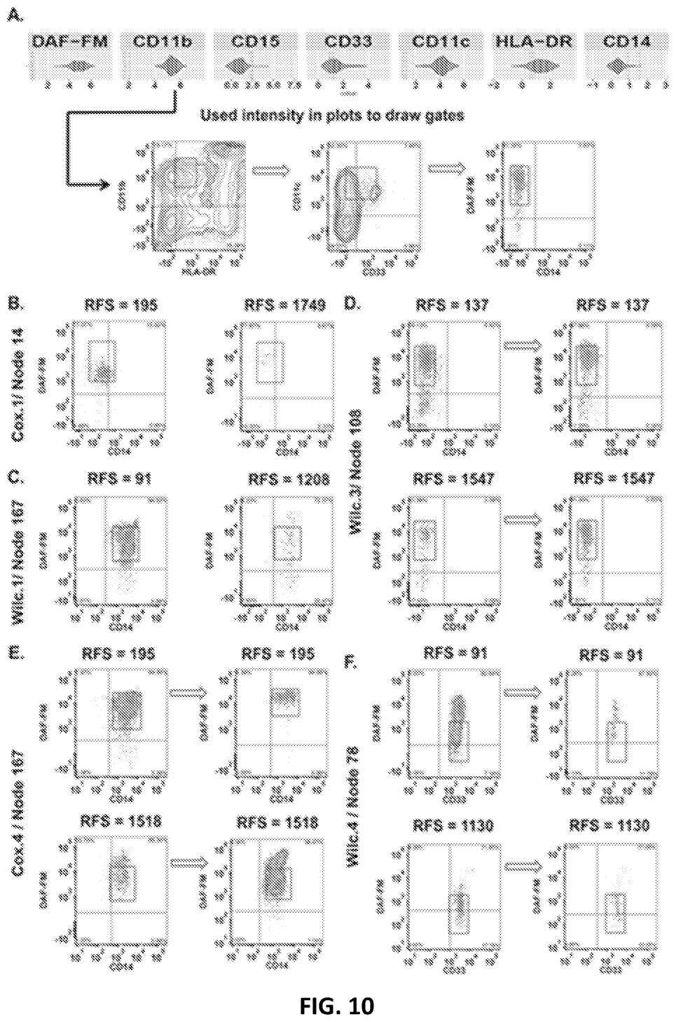

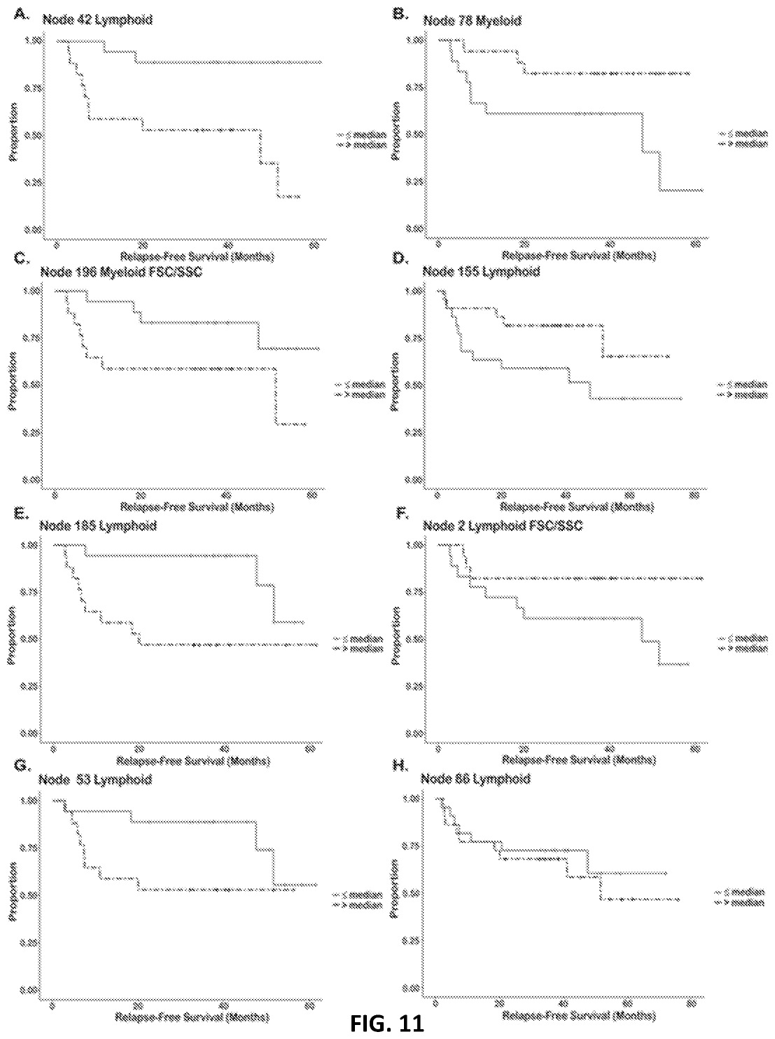

Nine lymphoid and 7 myeloid markers with and without the addition of scatter properties of the cells (forward scatter [FSC] and side scatter [SSC] areas) from the flow cytometry panels were used in the MPAT-R algorithm to delineate the phenotypes of specific cell populations. In the first step, the different phenotypes of cells were clustered using the SPADE algorithm (FIG. 8A). The second step was to visualize the clustering in a user-friendly way, as ascertaining the phenotypes via traditional clustering analysis is time consuming (FIGS. 8B-8C). Violin plots were constructed with positive/negative cut-off lines for each node marker (cell phenotype) in patient samples. The MPAT-R application can display the violin plots for each node (cell phenotype) (FIG. 8B) or for each sample (FIG. 8C). In addition, the application can scale the violin plots to the number of events in the node/sample. Each row is labelled by the node/sample number and the number of events in that node/sample (FIGS. 8B-8C). The third step is to perform phenotype dimension reduction (FIG. 2D). This process associates the number of events (cells) with each node (phenotype) to be used in downstream statistical analyses. After the phenotype dimension reduction, in which the multi-parameter flow cytometry stain is reduced to the number of events in a node for a particular sample, statistical analyses were used to determine which nodes were associated with RFS (FIG. 2E). The visualization tool allows the user to quickly ascertain the phenotype, using traditional flow cytometry software such as FCS Express. Nodes found in the statistical analyses were visualized using FCS Express 6, in which the fluorescence values were obtained from the violin plots and used for gating (FIGS. 9A and 10A).

Analysis Using the MPAT-R Algorithm

Four analyses were performed to determine which cell types may contribute to the effect of adjuvant ipilimumab treatment: 1) pre-treatment nodes associated with RFS (continuous analysis or stratified by RFS >1 year), 2) post-treatment nodes associated with RFS (continuous analysis or stratified by RFS >1 year), 3) pre-treatment nodes that changed after treatment but were not required to be associated with RFS, and 4) the number of events in a node that changed from pre-treatment to post-treatment that were associated with RFS (continuous analysis or stratified by RFS >1 year). The output was utilized as a score to determine which nodes were associated with RFS suitable for downstream analysis. The traditional analyses are now described, in which only the phenotypic markers are used for the clustering, in addition to the analyses that included the scatter properties of the cells (FSC and SSC) in the clustering tree.

Referring now to FIGS. 9A-9H, the integration of Multi-Dimensional Phenotype Analysis Tool in R (MPAT-R) output for lymphoid markers and delineation of cellular subsets associated with relapse-free survival (RFS) or treatment effects is described. Unsupervised gating using the cut-offs for each marker generated from the MPAT-R violin plots (as shown for node 42) to delineate cellular subsets not easily found in bivariate plots is shown in FIG. 9A. High pre-treatment levels of CD19⁺CD25^{-/lo} B cells (node 86=CD19⁺CD25^{-/lo}DAF-FM^{-/lo or +/lo}, hazard ratio=1.777, p=0.034) containing lower levels of nitric oxide (NO) were found primarily among patients with short-term RFS is shown in FIG. 9B. CD3⁺CD8⁺ effector T cells (node 155=CD3⁺CD8⁺CD25^{-/lo}DAF-FM⁺, RFS ≤1 year, mean=−8.666, SD=0.694; RFS >1 year, mean=−8.066, SD=0.956, p=0.035) with an intermediate NO level were found to be higher at the pre-treatment stage in patients with RFS >1 year is shown in FIG. 9C. Naïve or memory CD8⁺ T cells (node 53=CD3⁺CD8⁺CD127^{+/lo}DAF-FM⁺; hazard ratio=2.787, p=0.047) with moderate levels of NO were found to be more prevalent in the short-term RFS group post treatment is shown in FIG. 9D. A comparable CD8⁺ naïve or memory T-cell subset (node 150=CD3³⁰ CD8⁺CD127^{+ or +/lo} CD25^{-/lo} DAF-FM⁺, RFS ≤1 year, mean=−6.979, SD=0.583; RFS >1 year, mean=−7.421, SD=0.577, p=0.037) followed a similar pattern and was higher in RFS 1 year patients post treatment is shown in FIG. 9E. Interestingly, a subset of regulatory T cells (node 159=CD3⁺CD4⁺CD127^{-/lo}CD25⁺DAF-FM⁺, mean=−0.325, p=0.029) with a low-to-intermediate NO level presented a relative downward trend after treatment without any association with RFS is shown in FIG. 9F. CD11c⁺ natural killer cells (node 42=CD56^{+/lo}CD11c⁺CD25^{-/lo}DAF-FM⁺, hazard ratio=1.659, p=0.018) with an intermediate level of NO increased after treatment for most patients with short-term RFS, yet generally decreased after treatment for patients in the long-term RFS group is shown in FIG. 9G. A subset of rare CD4 CD8 double negative T cells (node 185=CD3⁺CD127^{+ or +/lo}CD25^{-/lo}DAF-FM⁺, RFS ≤1 year, median=0.510; RFS >1 year, median=−0.084, p=0.041) increased after treatment in a subset of short-term RFS patients and decreased in the majority of long-term RFS patients is shown in FIG. 9H.

After the preliminary analysis where all 200 nodes for each analysis (800 total for lymphoid and myeloid with/without FSC/SSC) were analyzed for phenotypes related to RFS in an unsupervised manner, all phenotypes with an p value <0.05 for each of the analyses were plotted in FCS Express for visualization purposes using batch techniques. Examples of relationships included: increases in the population of cells in melanoma patients prior to treatment with decreased RFS (FIG. 9B) or increased RFS (FIG. 9C). The same analysis was performed for immune cell populations post-treatment (FIG. 9D, 9E). Changes in the numbers of immune cell subsets were not associated with RFS (FIG. 9F). Lastly, changes in the numbers of immune cells with therapy were associated with RFS (FIG. 9G, 9H).

Referring now to FIGS. 10A-10F, the integration of the Multi-Dimensional Phenotype Analysis Tool in R (MPAT-R) output for the myeloid markers and delineation of cellular subsets associated with relapse-free survival (RFS) or treatment effects is described. Unsupervised gating performed through the use of the myeloid marker median fluorescence intensity measurements generated from MPAT-R violin plots (as shown for node 108) to delineate populations that were not easily seen within bivariate plots is shown in FIG. 10A. Pre-treatment, patients with short-term RFS were found to have higher concentrations of developing monocytes (node 14=HLA-DR⁺CD33^{-/lo or +/lo}CD11b⁺CD11c⁺CD14^{-/lo}DAF-FM⁺, hazard ratio=3.592, p=0.013) characterized by the low expression of the markers CD33, CD14 and moderate levels of nitric oxide (NO) is shown in FIG. 10B. Similarly, mature monocytes that are CD33⁺CD14⁺ (node 167=HLA-DR⁺CD33⁺CD11b⁺CD11c⁺CD14⁺DAF-FM⁺; RFS 1 year, mean=−8.394, SD=1.168; RFS >1 year, mean=−9.340, SD=1.125; p=0.028) with a moderate NO concentration were found to have an overall larger population before treatment among patients with RFS ≤1 year is shown in FIG. 10C. Dendritic cells (node 108=HLA-DR^{+/lo}CD33^{-/lo or +/lo or -/lo}CD11b⁺CD11c⁺DAF-FM⁺, mean=0.370, p=0.022) with intermediate levels of NO increased overall and were not associated with RFS is shown in FIG. 10D. Mature monocytes (node 167=HLA-DR⁺CD33⁺CD11b⁺CD11c⁺CD14⁺DAF-FM⁺; hazard ratio=0.488, p=0.021) with an intermediate level of NO, had an overall increase in the long-term RFS patients after treatment and decreased in short-term RFS patients after treatment is shown in FIG. 10E. Whereas, mature monocytes (node 78=HLA-DR⁺CD33⁺CD11b⁺CD11c⁺CD14^{+/lo}DAF-FM^{+/lo}; RFS ≤1 year, median=−1.289), RFS >1 year, median=−0.243; p=0.037) with low CD14 and NO expression decreased more in the short-term RFS group than in the long-term RFS group is shown in FIG. 10F.

Similar analyses were conducted for the myeloid cell populations (FIGS. 10A-10F). The phenotypes found in the unsupervised analysis along with the flow cytometry plots are presented for the lymphoid nodes (FIGS. 9A-9H, Table S3 and S4) and myeloid nodes (FIGS. 10A-10F, Tables S5 and S6).

Referring now to FIGS. 11A-11H Kaplan Meier survival curves illustrating types of relationships between immune cell subsets and relapse free survival (strata=≤median number of events/node, >median number of events/node) is described. Utilization of the number of immune cells as a cut-off for predicting response is shown in FIGS. 11A-11B. Node 42 Lymphoid-NK cells (post-pre) is shown in FIG. 11A. Node 78 Myeloid-Monocytes (post-pre) is shown in FIG. 11B. Number of immune cells known to be associated with biology but may not useful as a biological cut-off without additional information is shown in FIGS. 11C-11D. Node 196 Myeloid FSC/SSC-MDSC (post-pre) is shown in FIG. 11C. Node 155 Lymphoid-Effector T cells (pre) is shown in FIG. 11D. The survival curves separate after a prolonged period of time is shown in FIGS. 11E-11F. Node 185 Lymphoid-αβ T cells (post-pre) is shown in FIG. 11E. Node 2 Lymphoid FSC/SSC-Tregs (post-pre) is shown in FIG. 11F. The survival curves merge after a prolonged period of time indicating that relapse may be prolonged in these patients (Node 53 Lymphoid-CD8 memory/naïve T cells (post)) is shown in FIG. 11G. The survival curves show no difference, but there is a difference in the continuous variable that necessitate a different cut-off value for biomarker determination (Node 86 Lymphoid-B Lymphocytes (pre)) is shown in FIG. 11H.

Categories of nodes that were not studied further included the inability to visually discern the difference between responders and non-responders from the flow plots and nodes that contained less than a maximum of ^{˜}200 cells. Addition of FSC/SSC to the analysis did reveal additional nodes associated with RFS. Cell subsets such as myeloid-derived suppressor cells (MDSCs) and Tregs associated with RFS were delineated in the FSC/SSC clustering analysis (Node 196 and 2, respectively). The resulting immune cell phenotypes association with RFS may be split into four categories based upon the RFS characteristics for each node (strata=median number of events/node, >median number of events/node) as seen in the Kaplan Meier plots. First, there may be a cut-off that may be utilized for potential biomarkers (Node 42− NK cells positive for DAF-FM, FIG. 11A and Node 78-Monocytic cells positive/low for DAF-FM, FIG. 11B). Second, the variables (# of events in a node) may be continuous such that the immune cell phenotype present may be indicative of biology but may not be useful for exact cut-off biomarkers (Node 196 FSC/SSC-MDSC positive for DAF-FM, FIG. 11C; Node 155-Effector T cell positive for DAF-FM, FIG. 11D). A third category is when the Kaplan Meier curves separate after a certain period of time, or a group of patients appear to derive long term benefit (>2 years). Phenotypes in this category include Node 185-CD4⁻CD8⁻αβT cells positive for DAF-FM (FIG. 11E), node 2 FSC/SSC-Treg negative/low for DAF-FM (FIG. 11F) and node 53-CD8 naïve/memory T cells positive for DAF-FM (FIG. 11G). A fourth category includes cell phenotypes (nodes) that were associated with RFS with a continuous variable (# events/node) but the survival curves did not demonstrate a difference in RFS (Node 86-B lymphocytes with negative or very low levels of DAF-FM found prior to therapy, FIG. 11H).

Lymphoid Cell Subsets Associated with RFS or Treatment Effects

High pre-treatment levels of CD19⁺CD25^{-/lo} B cells with low/negative levels of DAF-FM (Table S3, FIG. 9B, node 86) were found in samples from patients with short-term 1 year) RFS. In contrast, a subset of NK cells (Table S4, FSC/SSC node 117=CD56⁺CD8⁺CD11c⁺DAF-FM⁺) expressing CD8, CD11c, and moderate levels of NO were found to be more prominent in samples from some long-term (>1 year) RFS patients before therapy. Likewise, CD3⁺CD8⁺ effector T cells (Table S3, FIG. 9C, node 155) with intermediate levels of DAF-FM were found to be higher at the pre-treatment stage in a subset of patients with RFS >1 year. Naïve or memory CD8⁺ T cells (Table S3, FIG. 9D, node 53=CD3⁺CD8⁺CD127^{+/lo}DAF-FM⁺) with moderate levels of NO were found to be more prevalent in the short-term RFS group after treatment. A similar CD8⁺ T-cell subset (Table S3, FIG. 9E, node 150; Table S4, FSC/SSC node 121) containing moderate levels of NO decreased in some patients with long-term RFS after treatment. Interestingly, a subset of regulatory T cells (Tregs; Table S3, FIG. 9F, node 159=CD3⁺CD4⁺CD127^{-/lo}CD25⁺DAF-FM⁺) with an intermediate NO level presented a relative downward trend without any association with RFS. A similar downward trend that was not associated with RFS was also seen in a subset of CD4 T cells with moderate levels of DAF-FM (Table S3, node 143=CD3⁺CD4⁺CD127⁺CD25^{-/lo}DAF-FM⁺). Whereas CD11c⁺ NK cells (Table S3, FIG. 9G, node 42) with intermediate levels of DAF-FM were found to increase after treatment for most patients with short-term RFS, they generally decreased after treatment for patients with long-term RFS. This trend was also found in 2 very similar cell subsets (Table S3, node 24=CD56⁺CD11c^{+/lo}DAF-FM⁺; Table S4, FIG. S3C, FSC/SSC node 148=CD56⁺CD11c⁺CD25^{- or =/lo}DAF-FM⁺). Notably, this study found the presence of a rare double-negative CD4⁻CD8⁻αβT cell (Table S3, FIG. 9H, node 185; Table S3, node 36) that increased after treatment in a subset of short-term RFS patients but decreased after treatment among most long-term RFS patients.

Myeloid Cell Subsets Associated with RFS or Treatment Effects

Pre-treatment, patients with short-term RFS tended to have higher concentrations of monocytes (Table S5, FIG. 10B, node 14) that were characterized by the low expression of markers CD33 and CD14 and moderate levels of DAF-FM. Similarly, mature monocytes that were CD33⁺CD14⁺ (Table S5, FIG. 10C, node 167=HLA-DR⁺CD33⁺CD11b⁺CD11c⁺CD14⁺DAF-FM⁺) with a moderate NO level were found in greater numbers before treatment in the short-term RFS group than in the long-term RFS group. Very few plasmacytoid DCs were found, but those seen tended to be found with intermediate NO levels in the pre-treatment samples from patients with RFS >1 year (Table S5, node 185). Several cell types that were identified in the myeloid panel were actually lymphoid cells expressing CD11b, CD11c, and HLA-DR and were clearly delineated when FSC/SSC were used in the clustering algorithm. There were several nodes that fit into this category on the basis of a lack of markers expressed for each node, as seen in Table S5 (nodes=41, 52, 70, 160, and 190) and Table S6 (FSC/SSC nodes=46, 57, 78, 107, and 140). Of note, the current assay uses all 5 lasers on the LSR-2 machine. DCs (Table S5, FIG. 10D, node 108) with intermediate levels of DAF-FM had an overall increase after treatment and no association with RFS. Monocytes (Table S5, FIG. 10E, node 167) with an intermediate level of NO after treatment had an overall increase in the long-term RFS group and a decrease in a few short-term RFS patients, although monocytes (Table S5, FIG. 10F, node 78=HLA-DR⁺CD33⁺CD11b⁺CD11c⁺CD14^{+/lo}DAF-FM^{+/lo}) with low-CD14 and NO expression decreased more greatly in a subset of the short-term RFS group than in the long-term RFS group. A subset of MDSCs (Table S6, FSC/SSC node 196) increased after treatment among the short-term RFS patients, whereas the same subset of MDSCs decreased among the long-term RFS patients.

In general, lymphoid and myeloid cell populations showed a stratification of NO levels. MPATR was able to perform automatic phenotyping in a high-throughput fashion and contributed to our understanding of why different NO-containing cell populations were associated with increased RFS in patients receiving adjuvant ipilimumab. This study was not statistically powered for subset analyses; therefore, to generate any one phenotype as a biomarker for response or failure to ipilimumab therapy, additional studies will be required. The strength of this MPATR algorithm is that it can clearly illustrate phenotypes to physicians/researchers in real time and be used in hypothesis-generating or -testing experiments.

### Discussion

An MPAT-R algorithm was developed to phenotype immune cell subsets in multi-dimensional flow cytometry experiments performed on a set of peripheral blood samples obtained from patients prior to and after adjuvant ipilimumab treatment with a peptide vaccine. This algorithm facilitates identification of the pro- and anti-tumor activities of NO, but equally as important, it has established a method by which multi-dimensional phenotyping results in the form of an intricate graphical description. This algorithm may be placed in the hands of physicians who without prior knowledge of advanced clustering techniques can make use of them for clinical trials. In addition, a table is provided to ascertain the relationship between the number of cells in the phenotype (node) and survival.

The output of the MPAT-R algorithm is a user-friendly computational analysis tool for the delineation of clinically relevant immune cell populations in the peripheral blood of patients receiving immunotherapy. The MPAT-R algorithm grants the user the ability to both cluster as well as visualize specific cellular subsets across patient populations in an easy-to-use interface, unlike other clustering or visualization algorithms, such as CCAST, Citrus, tSNE, and viSNE. This ability to see all of the markers simultaneously makes the algorithm beneficial for both researchers and clinicians to clearly visualize the phenotypes of immune cells. The generation of violin plots for each node permits the implementation of a defined gating strategy into flow cytometry analysis software such as FCS Express 6, thereby eliminating human bias and revealing clinically important cellular subsets. As an example, rare phenotypes such as a subset of CD8⁺ NK cells (Table S4, FSC/SSC node 117) and CD4⁻CD8⁻αβ T cells (Table S3, nodes 185 and 36) were easily found in an unsupervised manner. In addition, MPAT-R's events-per-node versus patient sample summary table output provides a quantitative construct of relative node density, thereby eliminating the subjective interpretation of relative node density based on SPADE trees. The summary table also allows the user to troubleshoot the data by finding batch effects and examining file integrity of the flow cytometry files and associated compensation files. Mathematical properties of k-means clustering results in either truncating zeros or nodes with extremely high values. Although identification of specific issues must be performed by those experienced with the relevant techniques, MPAT-R allows the novice to determine whether there is an issue with their dataset (truncation of data and inappropriate compensation matrix applied to the data), prompting them to obtain advice if needed. Other groups have attempted to develop user-friendly tools to visualize the output from SPADE clustering on a per-sample basis; however, MPAT-R algorithm is believed to be the only one to date that has the ability to quickly visualize a node's phenotype as well as a node's density across an entire population of patient samples. MPAT-R provides a user-friendly interface to delineate cell populations that may be important in clinical samples in longitudinal clinical studies. The algorithm was used in the current study to analyze the distribution of NO in immune cell subsets.

NO can show both pro- and anti-tumor effects in a concentration- and context-dependent manner. For instance, suppression of T cells by MDSC was found to be dependent on the MDSC's NO content. It was recently reported that MDSC-produced NO can interfere in the cancer cell antigen presentation from DCs to T cells via the Jak-STAT signal transduction pathway. On the other hand, NO production is also important for macrophage-mediated melanoma cell killing and plays a vital role in the regulation of T-cell functions, their differentiation, and cell death. In studies, different levels of NO have been observed in a wide variety of immune cells that are associated with increased or decreased RFS, depending on cell type. For instance, CD8⁺ NK cells and monocytes positive for DAF-FM staining were associated with anti-tumor activities at either pre-treatment or post-treatment stages. The numbers of effector T cells as a group changed with therapy but were not associated with RFS. However, as demonstrated in FIG. 9C (lymphoid node 155) there was one subset of effector T cells associated with RFS. On the other hand, intermediate levels of NO in B cells, double-negative αβ T cells, CD8⁺ naïve or memory T cells, MDSCs, immature monocytes, and DCs were pro-tumor in nature and associated with short-term RFS. Interestingly, the MDSC populations were easily found when FSC and SSC were taken into account by the clustering algorithm.

Another class of immune cells that have had contradictory reports as to whether they correlate with ipilimumab efficacy are Tregs. More recently, depletion of Tregs was found to be important in CD8⁺ T-cell-inflamed tumors. In addition, other recent studies have demonstrated overall decreases in Tregs after ipilimumab treatment, but there is significant overlap between the 2 groups. Two different Treg subsets were identified by employing the automated phenotyping algorithm MPAT-R: one with a moderate NO (lymphoid node 159) level and the other one with a low/negative NO level (node 2 FSC/SSC lymphoid). The moderate NO population changed after treatment, yet no correlation with response was shown. In contrast, the small subset of Tregs with low/negative NO levels was associated with longer term RFS after 2 years. Thus, by employing the MPAT-R algorithm with DAF-FM as an additional marker, a dichotomy between the two distinct Treg subsets in regards to how their NO levels correlated with RFS were identified. Similarly, attempts have been made to evaluate the biomarker potential of Teff, yielding no conclusive results to date. Using MPAT-R, it was found that one node of Teff cells with intermediate levels of NO are associated with increased RFS (lymphoid node 155), whereas Teff with low/negative NO level (lymphoid node 107) that had changed following treatment did not show any association with RFS. Interestingly, recent studies have demonstrated the importance of memory and NK cells in high-dimensional analysis, but the numbers overlapped between responders and non-responders. In this study, NK cells were associated with increased RFS, but NK subsets were also found that have no such relationship (Table S3). As expected, it was found that B cells associated with response have low levels of NO. It is possible that they may be serving a regulatory role, even though they do not express high levels of CD25. The ability of MPAT-R to distinguish cell subsets should be useful in future biomarker exploration studies containing larger patient cohorts for immune-based therapy.

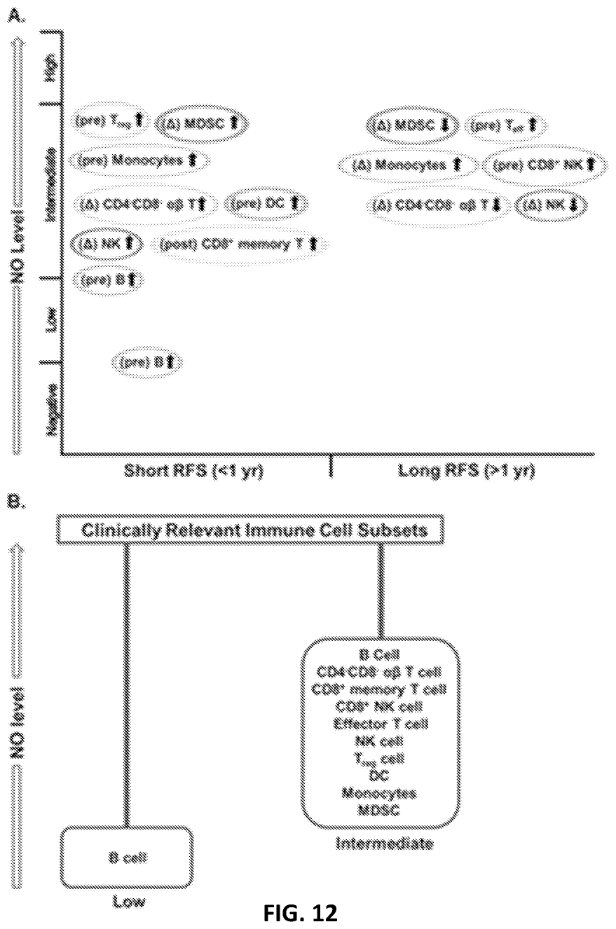

The MPAT-R algorithm also demonstrated a similar dichotomy in the role of NO for myeloid cells. Traditionally, monocytic accumulation in the tumor and blood has been associated with decreased survival. More recently, peripheral blood monocyte levels have been found to overlap between responders and non-responders in stage IV melanoma patients. This overlap was observed, but levels of NO may distinguish different cell populations (FIGS. 12A-12B). For instance, monocytes with negative or low levels of NO (myeloid FSC/SSC node 185) were only associated with treatment changes, whereas monocytes with intermediate levels of NO (myeloid node 78) were associated with increased RFS. Lastly, MDSCs are immature myeloid cells that function to inhibit other immune cells, such as lymphoid cell populations. Treatment with ipilimumab has been shown to decrease a subset of MDSC (monocytic) and increase CD8 memory T cells. MDSC followed this trend of increasing in patients who had shorter-term RFS.

Referring now to FIGS. 12A-12B, the dichotomous role for nitric oxide (NO) in pro- and anti-tumor effects is described. As described herein, four areas related to how the levels of NO change in immune cell subsets with therapy were investigated: 1) pre-treatment nodes associated with relapse-free survival (RFS; continuous analysis or stratified by RFS >1 year), 2) post-treatment nodes associated with RFS (continuous analysis or stratified by RFS >1 year), 3) pre-treatment nodes that changed after treatment but were not required to be associated with RFS, and 4) the number of events in a node that changed with treatment that were associated with RFS. Cell subsets with interesting clinical trends associated with low or intermediate NO levels is shown in FIG. 12B. Association of these same cell subsets with RFS and NO levels is shown in FIG. 12A. In FIGS. 12A and 12B, the following abbreviations are used: DC, dendritic cell; MDSC, myeloid-derived suppressor cell; NK, natural killer cell; RFS, relapse-free survival; T_{reg}, T-regulatory cell.

MPAT-R is a tool that can be used by physicians to profile phenotypes among immune cell populations. With this dataset, NO can be detected in distinct immune cell populations that are associated with RFS. The same type of analysis may be performed on other patient datasets to decipher the immune cell milieu of both the peripheral blood and, potentially, also the tumor microenvironment. It is believed that the approach to the analysis, which has revealed trends demonstrating the dichotomy of NO associated with pro-/anti-tumor effects to immune-based therapy, is relevant to the translational medicine community at large and may be readily applied to clinical trials by allowing for efficient unsupervised organization of immune cell phenotypes.

Materials and Methods

Patient Samples

Seventy-nine cryopreserved PBMC samples from patients with resected stage IIIc/IV melanoma were provided by Moffitt Cancer Center. Patients were treated with ipilimumab (3 to 10 mg/kg every 6 to 8 weeks for 12 months) and 3 separate subcutaneous vaccine injections, as previously described in the clinical trial publication. The current analyses used matched samples from 35 of these patients that were taken before and about 13 weeks after ipilimumab treatment initiation, 9 unmatched samples that were collected from melanoma patients before immunotherapy, and 7 PBMC samples that were isolated from normal/healthy individuals. The clinical responses (RFS and overall survival) of these patients were recorded in the primary clinical trial study. Collection and handling of all human biological samples were conducted by following the ‘good clinical practice’ (GCP) guidelines.

Flow Cytometric Analysis of Peripheral Blood Samples

PBMCs were obtained from the blood samples by ficol density-gradient centrifugation. Patient samples were available from leukapheresis specimens collected at the time of the clinical trial. Frozen PBMCs were used in this retrospective study. Two flow cytometry panels were constructed: myeloid and lymphoid. PBMCs were stained with the antibodies, after proper titration to obtain an optimal signal-to-noise ratio (myeloid panel: DAF-FM [NO marker; Fisher, Hampton, Mass.], HLA-DR-PE-Cy7, CD33-APC, CD11b-BV421, CD14-BUV395, CD15-BV510, and CD11c-PE [BD Biosciences, San Jose, Calif.]; lymphoid panel: DAF-FM, along with CD3-BUV395, CD8-BV510, CD11c-PE, CD56-BV421 [BD Biosciences] and CD4-AF700, CD19-PE-Dazzle, CD25-PE-Cy7, CD127-APC [Biolegend, San Diego, Calif.]). Dead cells were excluded with Zombie NIR (BioLegend) staining. Data acquisition (100,000 live events) was performed by using an LSRII flow cytometer (BD Biosciences) and immunophenotypic analysis by FCS Express 6 software (De Novo Software). Proper gating was set with fluorescence-minus-one and antibody isotype controls. Rainbow fluorescent particles (BD Biosciences) were also used to calibrate the cytometer correctly between all runs, and flow cytometric compensation beads (Fisher) were used to establish robust compensation matrices.

Measurement of pSTAT1

Frozen PBMCs were thawed in a water bath at 37° C., washed to remove the freezing media, and allowed to rest overnight in complete media at 5% CO₂ at 37° C. Stimulation with IFNα is accomplished by replacing the resting media with fresh media containing various concentrations of IFNα and incubating for 30 minutes. The live/dead marker Zombie NIR (Biolegend, San Diego, Calif.) was used prior to permeabilization to prevent inappropriate uptake of the dye. After live/dead staining and wash, the samples were permeabilized using the FIX PERM cell permabilization kit methanol modification (Fisher, Hampton, Mass.). In short, the cells were fixed and preserved while stored at −20° C. for a minimum of 2 hours then permeabilized for pStat1 staining. Phospho-Stat1-AF488 (BD Biosciences, San Jose, Calif.) was applied while the cells were being permeabilized for 1 hour at room temperature. Samples were read on an LSR II flow cytometer, and 100,000 live cell events were recorded. Controls included: flow cytometric compensation beads (Fisher) to establish robust compensation matrices, fluorescence-minus-one controls to set negative and positive gates, and isotype controls for patient variations.

Analyses

Nine pre-treatment only and 35 matched pre-/post-adjuvant ipilimumab and vaccine treatment PBMC samples from patients with resected stage IIIc/IV melanoma were available for statistical analyses. Another 7 PBMC samples from individuals without disease were also collected and used as a “normal” quality control population in each run. In total, there were 44 pre-treatment samples, 35 post-treatment samples, and 7 normal samples. The output from the first step of the analysis (SPADE) created 200 nodes for the 2 different flow cytometry panels (lymphoid, myeloid). A second clustering analysis generated 200 nodes (cell populations) in which the FSC and SSC areas were used as additional clustering parameters. The cell populations were normalized by total number of cells per sample and analyzed in log 2 scale before application to parametric tests. Combat, a de-batching method, was performed to remove potential experimental batch effects and was followed by visual confirmation using a principal component analysis. To identify which populations (nodes) were associated with RFS, 2 sets of analyses were performed: Cox proportional-hazard model (Cox regression) and Wilcoxon rank sum test. Cox regression was performed to evaluate cell populations associated with RFS. RFS was defined as the time from study enrollment to time of relapse and was censored at the last clinic appointment. Wilcoxon rank sum test was the second analysis. The progression status for each patient was based on a clinically relevant empirical definition 1 year RFS versus >1 year RFS). It was investigated whether either pre-treatment level, post-treatment level, or the level of change of each cell population differed between patients with disease relapse and those without. To identify whether therapy alone alters percentages of immune cells in the peripheral blood, the Wilcoxon rank sum test was used. Statistical analyses were performed in the program Rstudio. The output was utilized as a score to determine which nodes were associated with RFS suitable for downstream analysis. The same analyses were performed for the datasets obtained, using FSC and SSC in the clustering algorithm.

### Example 4

In 2018, there are projected to be 91,270 cases of melanoma with 9,320 deaths in the United States. Florida shares a major burden of this disease with 6,614 cases reported in 2015. In the past few years, melanoma therapy has undergone a revolution with therapies that target the immune system. Pembrolizumab and nivolumab are currently the two FDA approved anti-PD-1 antibodies (immune based agents) for the treatment of metastatic melanoma. However, even with these advancements in immune based-therapies upwards of 60% of people will not respond. Thus, new strategies are needed to improve therapeutic outcome.

The mRNA and proteomics multi-omics analysis on a preliminary series of 30 and 19 FFPE samples, respectively collected from patients prior to anti-PD-1 therapy has been completed and demonstrated that antigen presentation, NO-dependent pathways among other pathways are upregulated in patients who respond to treatment.

Determination of PD-L1⁺SOX10⁺ Melanoma Cells in Proximity to T Cells as a Measure of Response to Anti-PD-1 Therapy

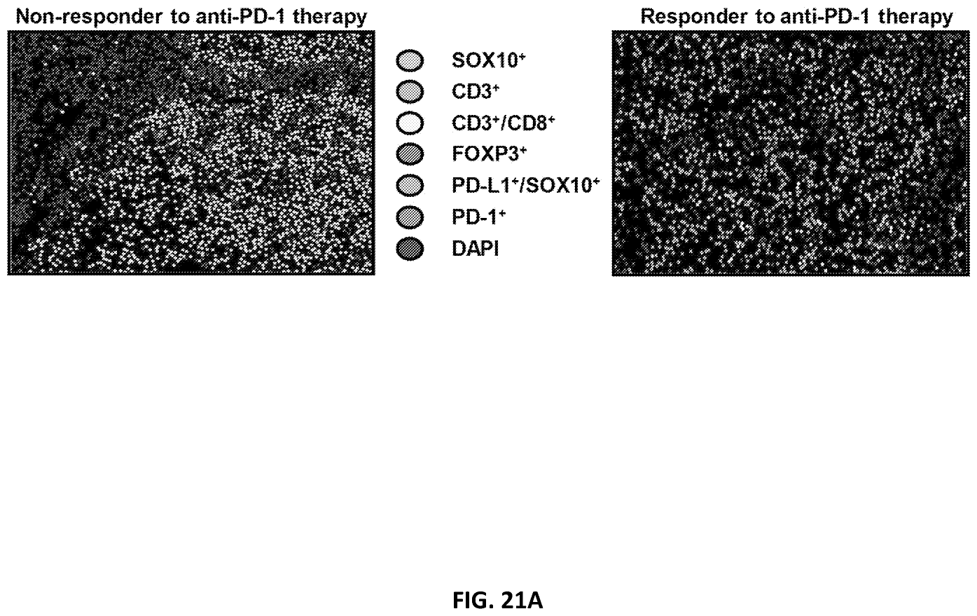

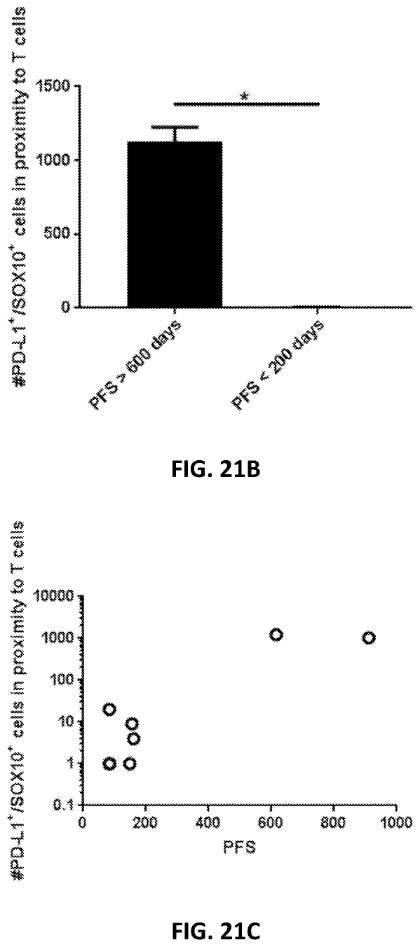

In preliminary data (FIGS. 21A-21C), it was demonstrated that PD-L1⁺SOX10⁺ melanoma cells are found in increased proximity to T cells in those patients that are responding to anti-PD-1 therapy. This relationship in FFPE sections obtained prior to therapy from a group of 26 patients undergoing anti-PD-1 therapy is analyzed to test this relationship in a larger group of patients.

For the preliminary data set 10 FFPE tissue samples collected prior to anti-PD-1 therapy were immunostained using the PerkinElmer OPAL™ 7-Color Automation IHC kit (Waltham, Mass.) on the BOND RX autostainer (Leica Biosystems, Vista, Calif.). The OPAL 7-color kit uses tyramide signal amplification (TSA)-conjugated to individual fluorophores to detect various targets within the multiplex assay which in the experiments includes PD-L1, CD3, SOX10 (melanoma antigen), PD-1, CD4, CD8, FOXP3, and DAPI. Sections were baked at 65° C. for one hour then transferred to the BOND RX (Leica Biosystems). All subsequent steps (ex., deparaffinization, antigen retrieval) were performed using an automated OPAL IHC procedure (PerkinElmer). OPAL staining of each antigen occurred as follows: slides were blocked with PerkinElmer blocking buffer for 10 min then incubated with primary antibody at optimized concentrations followed by OPAL HRP polymer and one of the OPAL fluorophores. Individual antibody complexes are stripped after each round of antigen detection. After the final stripping step, DAPI counterstain is applied to the multiplexed slide and is removed from BOND RX for coverslipping. Autofluorescence slides (negative control) were included, which use primary and secondary antibodies omitting the OPAL fluors. All slides were imaged with the Vectra®3 Automated Quantitative Pathology Imaging System and analyzed on the InForm software (Perkin Elmer).

A two-sample t-test can be used to compare the levels of PD-L1⁺/SOX10⁺ melanoma cells in proximity to T cells in the same Region of Interest (ROI+2 mm²) between tissue slices obtained from patients who responded to anti-PD-1 therapy compared to those that did not respond. Assuming a response rate of 40% after anti-PD-1 treatment and a standard deviation of 1,000 for the level of PD-L1⁺/SOX10⁺, a total sample size of 26 is required to achieve at least 90% power when a mean difference is 1,000 between the responders (n=11) and non-responders (n=15). For a multiple test correction, an overall adjusted significance level of 0.05 (0.01 for each of five ROIs) can be utilized using the Bonferroni method. Descriptive statistics for the numbers of other types of immune cells (CD8⁺ T cells, T-regulatory cells etc.) can be calculated across tumor regions with different immune phenotypes to characterize tumor immune environments. As a secondary analysis, survival analysis can be performed to examine association between marker level (i.e. #PD-L1+SOX10⁺ cells in the vicinity of T cells) and progression free survival/overall survival point using Cox regression model and log-rank tests (when the marker level is dichotomized) as the patient follow-up information becomes mature at the end of the study.

Referring now to FIGS. 21A-21C, patients responding to anti-PD-1 have increased expression of PD-L1 on melanoma tumor cells (SOX10+) and infiltrating T cells is described. The levels of PD-L1/SOX10 in proximity to T cells in the same region of interest on the slide (ROI) is elevated prior to therapy in the patients who respond to anti-PD-1 therapy versus those that do not respond to this immune therapy. For example, FIG. 21A compares the levels of PD-L1/SOX10 in proximity to T cells in the same region of interest for a non-responder to anti-PD-1 therapy (left side) and a responder to anti-PD-1 therapy (right side). Tissues samples from 26 melanoma patients undergoing anti-PD-1 therapy will be subjected to this assay.

PD-L1⁺SOX10⁺ melanoma cells in proximity to T cells in 10 FFPE sections from patients with metastatic melanoma prior to anti-PD-1 therapy demonstrates this combination of cells in the tumor microenvironment is markedly elevated in patients responding to therapy. This cohort was pretreated with a number of agents including ipilimumab which accounts for the 20% response rate and the PFS is illustrated in the graphs below. It should be noted that both of the patients in the long PFS group were censored at the last follow up, but all 8 patients in the low PFS group progressed on anti-PD-1 within 6 months of initiation of treatment (FIG. 21B; p<0.001; unpaired T test). It is well accepted in the literature that patients benefit from anti-PD1 have at least 6-12 months of progression free survival even without an objective response. If it is possible to consistently predict those patients who are going to do very well and very poorly then this would be useful information for the patient population as those not predicted to respond should consider other therapeutic options. In FIG. 21C, the data is plotted using a log 10 transformation. Three of these data points are not depicted on this graph as they were 1 (N=2) or 0 (N=1) prior to transformation. This panel will be utilized on FFPE tissues derived from patients undergoing anti-PD-1 treatment. The FFPE multi-plex immune panel will also allow characterization of the architecture of the tumor microenvironment. 26 samples from patients prior to undergoing anti-PD-1 will be measured.

Analysis of Multidimensional Flow Cytometry to Determine Level of Immune Suppression/Stimulation in Patients Undergoing Anti-PD-1 Therapy

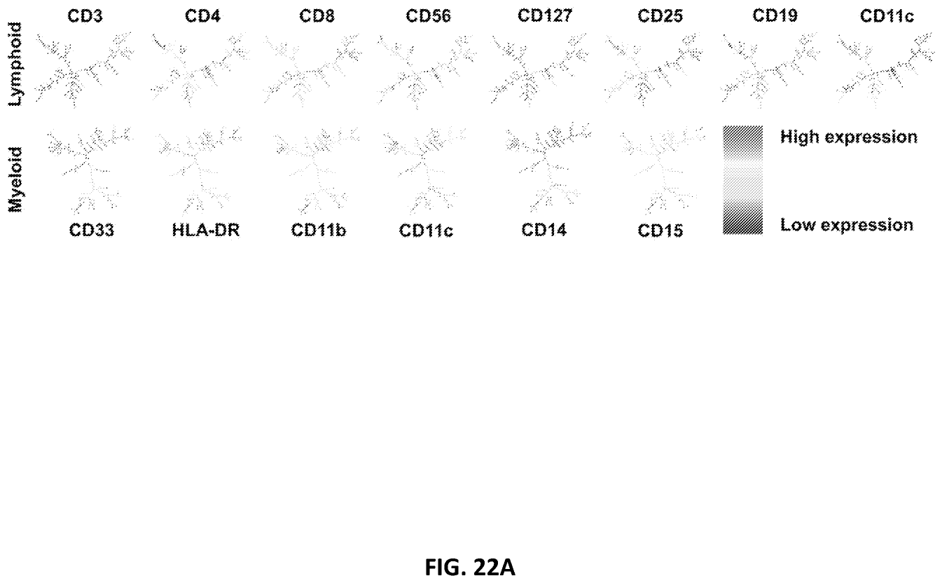

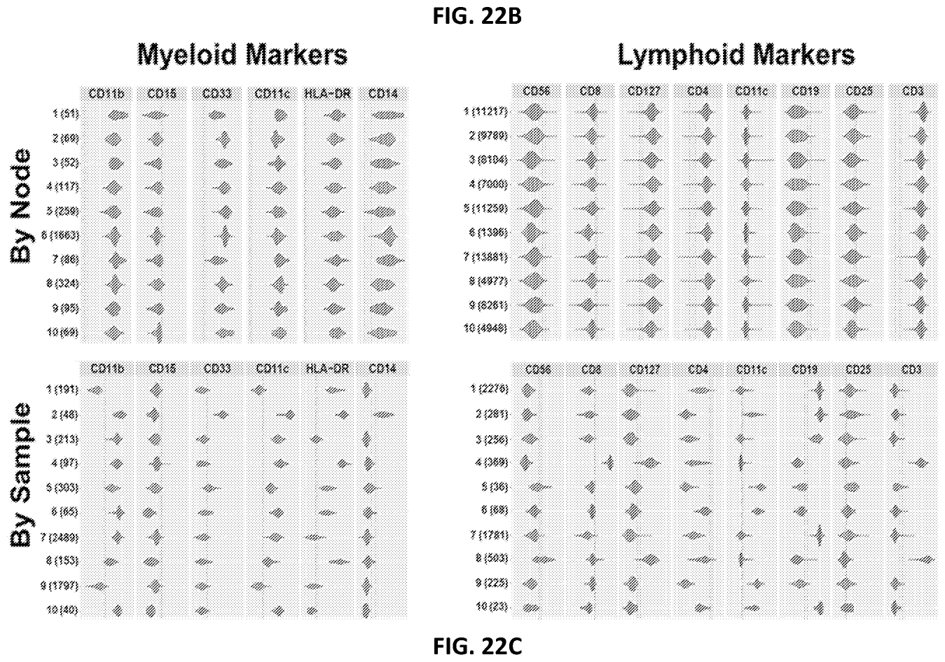

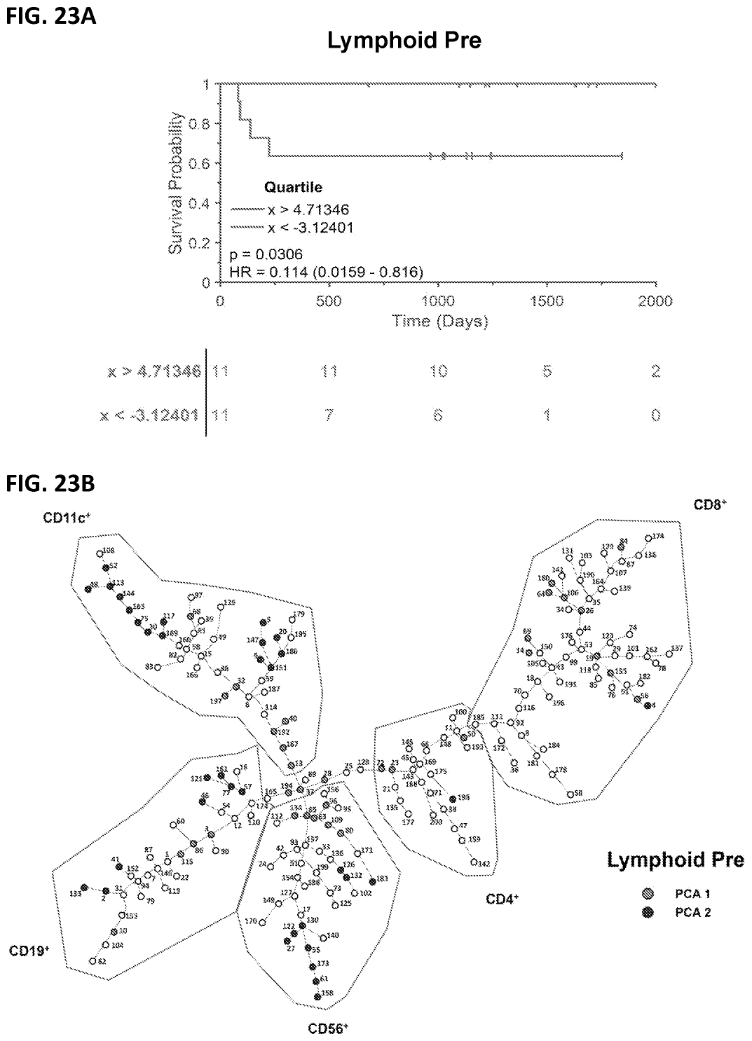

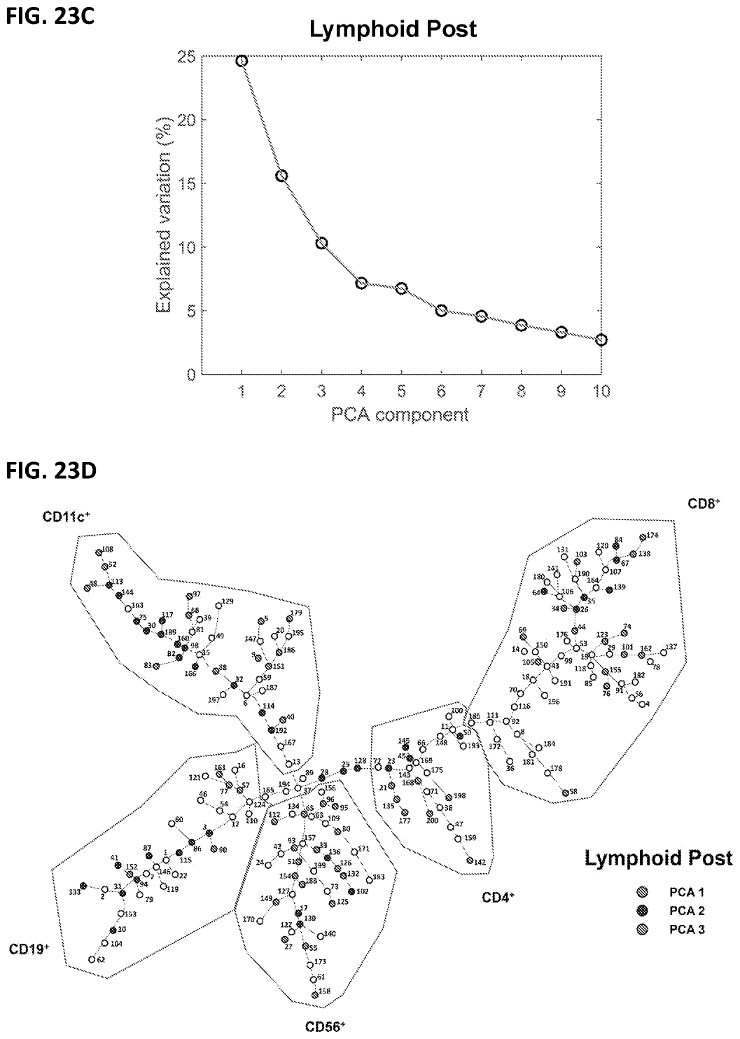

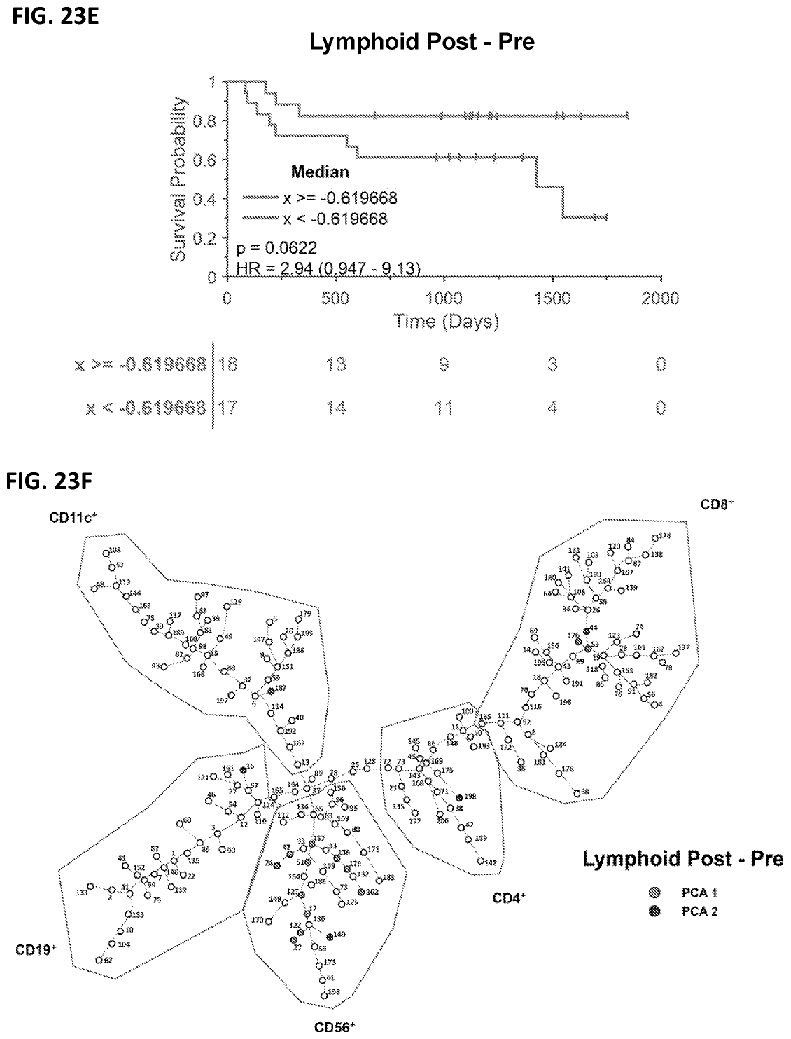

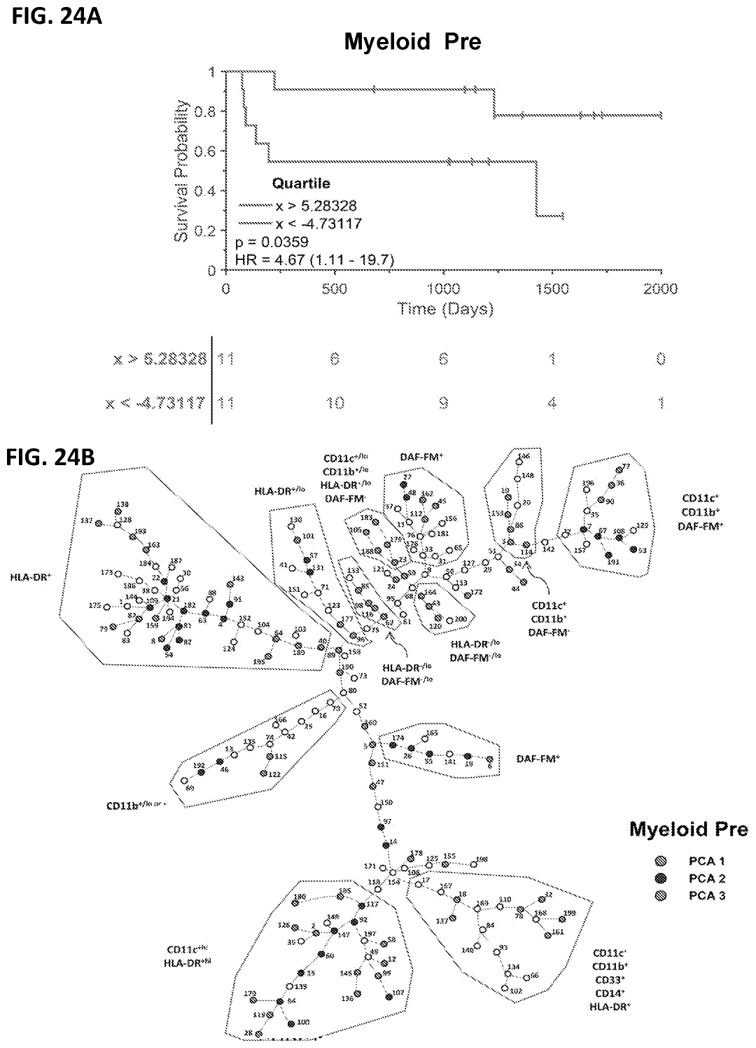

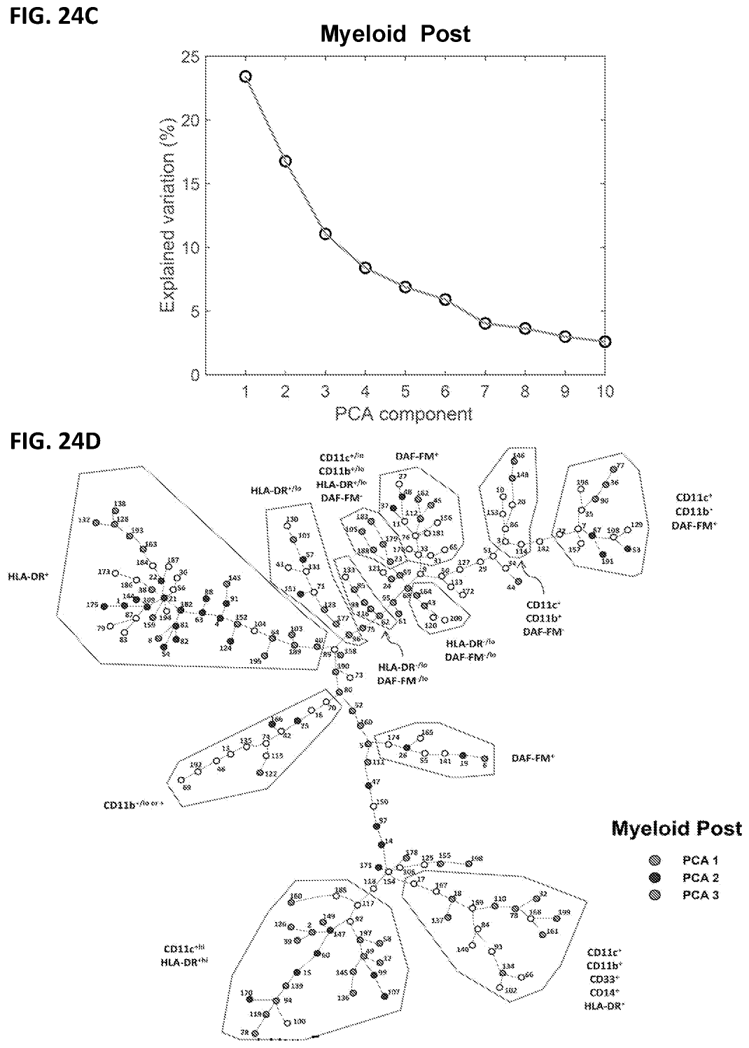

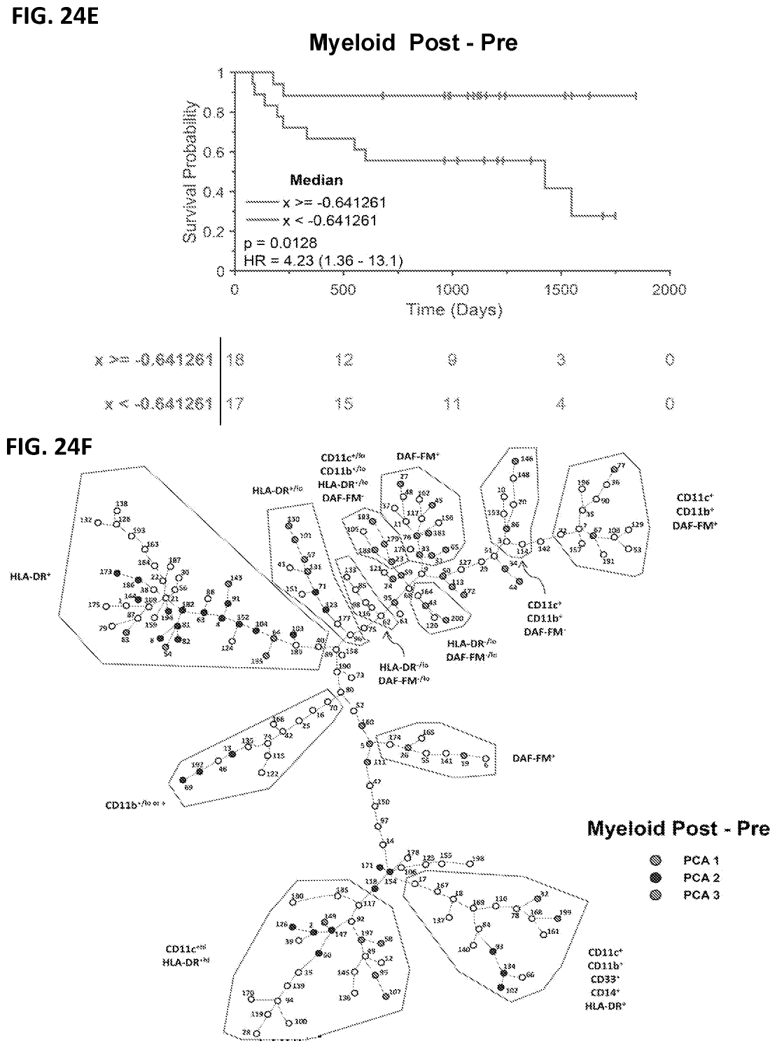

Referring to FIGS. 22A-22C, 8 lymphoid and 6 myeloid markers (in addition to a live dead stain) with and without the addition of scatter properties of cells (forward scatter area and side scatter area) from flow cytometry panels were used in the MPATR algorithm, to delineate the phenotypes of specific cell populations is described. In the first step, the different phenotypes of the cells were clustered using the SPADE algorithm, as shown by the SPADE trees in FIG. 22A. These panels are specifically utilized for clinical samples given the potential importance of effector T cells, helper T cells, NK Cells, T regulatory cells (Tregs), monocytic cells and myeloid derived suppressor cells (MDSC). In the second step, a tool to visualize the phenotypes with positive/negative cutoffs. FIGS. 22B and 22C are the violin plots for each node (cluster) (FIG. 22B) or for each sample (FIG. 22C). In addition, the application can scale the violin plots to the number of events in the node/sample. Each row is labelled by the node/sample number and the number of events in that node/sample. The numbers of MDSC may decrease while the numbers of cells such as CD8 effector T cells or non-regulatory CD4 T cells may increase in those patients responding to anti-PD-1 therapy. In the third step, phenotype dimension reduction is performed (see FIG. 2D). This process associates the number of events (cells) with each node (phenotype) to be used in downstream statistical analyses. This process simplifies the data to a 2×2 table that can be associated with survival outcomes

As discussed herein, a method for measuring phenotypes from high dimensional flow cytometry experiments has been developed (e.g., the MPAT-R algorithm). In the first step, the different phenotypes of cells were clustered using the SPADE algorithm (FIG. 22A). The second step was to visualize the clustering in a user-friendly way, as ascertaining the phenotypes via traditional clustering analysis is time consuming (FIGS. 22B-22C). Violin plots were constructed with positive/negative cut-off lines for each node marker (cell phenotype) in patient samples. The MPATR application can display the violin plots for each node (cell phenotype) (FIG. 22B) or for each sample (FIG. 22C). In addition, the application can scale the violin plots to the number of events in the node/sample. Each row is labelled by the node/sample number and the number of events in that node/sample (FIGS. 22B and 22C). The third step is to perform phenotype dimension reduction (see FIG. 2D). This process associates the number of events (cells) with each node (phenotype) to be used in downstream statistical analyses. After the phenotype dimension reduction, in which the multi-parameter flow cytometry stain is reduced to the number of events in a node for a particular sample, statistical analyses were used to determine which nodes were associated with RFS (see FIG. 2E). The visualization tool allows the user to quickly ascertain the phenotype, using traditional flow cytometry software such as FCS Express. Nodes found in the statistical analyses were visualized using FCS Express 6, in which the fluorescence values were obtained from the violin plots will be utilized for gating the cell populations. This analysis has been applied to measuring immune cell subsets in patients undergoing ipilimumab and can be readily applied to phenotype immune cells derived from peripheral blood of patients undergoing anti-PD-1 therapy.

PBMCs can be derived from the blood samples by ficol density-gradient centrifugation. Myeloid derived suppressor cells (MDSCs) are a major source of immune inhibition in cancer patients and melanoma patients in particular. MDSCs within the peripheral blood of melanoma patients will be phenotyped using a flow cytometric technique that employs fluorescently-labeled antibodies (with compatible fluorochromes) for CD33, CD11b, CD11c, HLA-DR, CD14 (monocytic marker), and CD15 (granulocytic marker). Although MDSCs are one of the major inhibitory cell types, other immune subsets and a normal donor control PBMCs can be characterized in each experiment. T cell subsets, DC subsets, NK cells and B cells within the peripheral blood of melanoma patients will also be phenotyped using antibodies for CD4, CD8, CD11c, CD127, CD25, CD19, and CD56. A sense of the immune capacity of myeloid and lymphoid cells of those cells found to be in the blood of those patients responding or resistant to anti-PD-1 can be obtained by measuring via flow cytometry immunosuppressive (PD-L1, Arginase 1, Reactive Oxygen Species) and immunostimulatory (CD69, TCR-zeta, CD103, and intracellular IFN-gamma) molecules on those immune cells found to be important with response or resistance in our sample cohort. Cells can be stained, fixed in 1% paraformaldehyde, and analyzed on an LSR II flow cytometer (100,000 live events) using standard gates, isotype control antibodies and compensation beads (Invitrogen, Waltham, Mass.) to establish criteria for positive staining and compensation controls. Dead cells can be excluded with Zombie NIR (BioLegend) staining. Data acquisition (100,000 live events) can be performed by using an LSRII flow cytometer (BD Biosciences; with potential to expand to the BD Symphony in the core facility) and immunophenotypic analysis by FCS Express 6 software (De Novo Software). Proper gating can be set with fluorescence-minus-one and antibody isotype controls. Rainbow fluorescent particles (BD Biosciences) can be used to calibrate the cytometer correctly between all runs, and flow cytometric compensation beads (Fisher) were used to establish robust compensation matrices. The percentage of positively-staining cells and their mean fluorescence intensity (MFI) can be calculated for cell populations of interest and the data will be processed using FCS Express software (Glendale, Calif.) and the MPATR algorithm.

The output from the first step of the analysis (SPADE) creates ^{˜}200 nodes for the flow cytometry panels (lymphoid, myeloid). The cell populations can be normalized by the total number of cells per sample and analyzed in log 2 scale before application to parametric tests. Combat, a de-batching method within the R statistical program, can be performed to remove potential experimental batch effects and can be followed by visual confirmation using a principal component analysis. To identify which populations (nodes) are associated with progression free survival (PFS), 2 sets of analyses can be performed: Cox proportional-hazard model (Cox regression) and Wilcoxon rank sum test. Cox regression can be performed to evaluate cell populations associated with PFS. PFS can be defined as the time from study enrollment (or start of anti-PD-1 therapy) to time of relapse and can be censored at the last clinic appointment. Wilcoxon rank sum test was the second analysis. The progression status for each patient will be based on a clinically relevant empirical definition (≤1 year PFS versus >1 year PFS). Pre-treatment level, post-treatment level, or the level of change of each cell population differed between patients with disease relapse and those without can be investigated. To identify whether therapy alone alters percentages of immune cells in the peripheral blood, the Wilcoxon rank sum test can be utilized. Statistical analyses will be performed in the program Rstudio. The output can be utilized as a score to determine which nodes (phenotypes) are associated with increased PFS suitable for combination with other data sets.

### Example 5

Example lymphoid and myeloid panels are shown below. This disclosure contemplates performing the lymphoid and myeloid panels using assay techniques known in the art including, but not limited to, Live/Dead control.

Lymphoid Tubes
1. CD11c
2. CD3
3. CD4
4. CD8
5. CD25
6. CD127
7. CD19
8. CD56
9. CD69
10. PD-L1
11. CTLA-4
12. CD3z
13. FOXP3
14. Arginase I
15. IFN-γ

Myeloid Tubes
1. CD11c
2. CD11b
3. CD33
4. HLA-DR
5. CD14
6. CD15
7. PD-L1
8. CTLA-4
9. FOXP3
10. Arginase I
11. IFN-γ

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for quantitatively predicting a cancer patient's response to immune-based or targeted therapy, comprising:
receiving patient data for the cancer patient, wherein the patient data is derived from a blood or tissue sample;
clustering a plurality of immune cell phenotypes present in the patient data, wherein the clustered patient data comprises a plurality of nodes;
generating a plurality of violin plots of signal intensity for at least one of the immune cell phenotypes, wherein each of the violin plots captures a number of events; and
statistically analyzing the violin plots to predict the cancer patient's response to immune-based or targeted therapy.

2. The method of claim 1, wherein statistically analyzing the violin plots comprises statistically analyzing the number of events in each node of the clustered patient data.

3. The method of claim 1, further comprising using the statistical analysis of the violin plots to detect a variation in at least one of the immune cell phenotypes present in the patient data.

4. The method of claim 1, further comprising using the statistical analysis of the violin plots to determine which of the nodes of the clustered patient data are associated with response to immune-based or targeted therapy.

5. The method of claim 1, further comprising using the statistical analysis of the violin plots to determine which of the nodes of the clustered patient data are associated with non-response to immune-based or targeted therapy.

6. The method of claim 1, wherein the statistical analysis is at least one of a principal component analysis, a cluster analysis technique, a distance matrix analysis, a Cox regression analysis, or a Wilcoxon signed-rank test.

7. The method of claim 1, wherein the violin plots are generated for each of the nodes of the clustered patient data, and wherein each of the violin plots captures the number of events per sample.

8. The method of claim 1, wherein violin plots are generated for the blood or tissue sample, and wherein each of the violin plots captures the number of events per node.

9. The method of claim 1, further comprising generating a graphical display of at least one of the clustered patient data or the violin plots.

10. The method of claim 1, further comprising recommending an immunotherapy or targeted therapy for the cancer patient that is predicted to respond to immune-based or targeted therapy.

11. The method of claim 1, wherein clustering a plurality of immune cell phenotypes present in the patient data comprises differentiating between cell populations based on a specific marker.

12. The method of claim 11, wherein the specific marker is nitric oxide (NO).

13. The method of claim 1, wherein the immune cell phenotypes present in the patient data are clustered using at least one of a spanning-tree progression analysis of density-normalized events (SPADE) algorithm, a t-Distributed Stochastic Neighbor Embedding (t-SNE) algorithm, a partitioning algorithm, a hierarchical clustering algorithm, a fuzzy clustering algorithm, a density-based clustering algorithm, or a model-based clustering algorithm.

14. The method of claim 1, wherein the patient data comprises at least one of flow cytometry data, immunoassay data, microscopy image data, mass spectrometry data, mass cytometry data, or genomic data.

15. The method of claim 1, wherein the immune cell phenotypes comprise myeloid markers.

16. The method of claim 15, wherein the myeloid markers comprise at least one of HLA-DR, CD33, CD16, CD44, CD66, Cd1c, CD83, CD141, CD209, MHC II, CD123, CD303, CD304, CD34, CD90, CD68, CD163, CD64, CD49d, 2D7 antigen, CD123, CD203c, FcεRIg, CD193, EMR1, Siglec-8, PD-1, PD-L1, Tim3, CD138, CD45, CD117, CD11b, CD34, CD36, CD64, CD61, CD117, CD62L, CD14, CD15, CD11c, CD103, DAF-FM, CTLA-4, FOXP3, Arginase I, or IFN-γ.

17. The method of claim 1, wherein the immune cell phenotypes comprise lymphoid markers.

18. The method of claim 17, wherein the lymphoid markers comprise at least one of CD3, CD3z, CD4, CD8, CD56, CD25, CD69, CD138, CD27, CD44, NKG2D, NKp30, NKp46, NKp46, CTLA-4, LaG-3, PD-1, TIM-3, PD-L1, CD45RA, CD45RO, CD62L, CD69, CD127, CD19, CD11c, CCR7, CTLA-4, DAF-FM, CTLA-4, FOXP3, Arginase I, or IFN-γ.

19. The method of claim 1, wherein the cancer patient has melanoma.

20. The method of claim 1, wherein statistically analyzing the violin plots comprises detecting variation in a node of the clustered patient data with respect to a data set, wherein the data set comprises respective patient data for a plurality of patient before and after administration of immune-based or targeted therapy.

21. The method of claim 20, further comprising adding the patient data for the cancer patient to the data set.

22. A method for treating a cancer patient, comprising:
predicting the cancer patient's response to immune-based or targeted therapy according to claim 1; and
administering an immunotherapy or targeted therapy to the cancer patient that is predicted to respond to immune-based or targeted therapy.

23. A system for quantitatively predicting a cancer patient's response to immune-based or targeted therapy, comprising:
a processor; and
a memory operably coupled to the processor, the memory having computer-executable instructions stored thereon that, when executed by the processor, cause the processor to:
receive patient data for the cancer patient, wherein the patient data is derived from a blood or tissue sample;
cluster a plurality of immune cell phenotypes present in the patient data, wherein the clustered patient data comprises a plurality of nodes;
generate a plurality of violin plots of signal intensity for at least one of the immune cell phenotypes, wherein each of the violin plots captures a number of events; and
statistically analyze the violin plots to predict the cancer patient's response to immune-based or targeted therapy.
